(19) United States
(12) Patent Application Publication
(10) Pub. No.: US 2012/0094281 A1
(43) Pub. Date: Apr. 19, 2012

(54) ARTICLES WITH SHELL STRUCTURES INCLUDING A CELL EXTRACTANT AND BIODETECTION METHODS THEREOF

(76) Inventors: Raj Rajagopal, Woodbury, MN (US); Smarajit Mitra, West St. Paul, MN (US); Matthew D. Reier, Beaverton, OR (US)

(21) Appl. No.: 13/266,490

(22) PCT Filed: May 6, 2010
(86) PCT No.: PCT/US10/33805

§ 371 Date: Dec. 21, 2011

Related U.S. Application Data
(60) Provisional application No. 61/175,996, filed on May 6, 2009.

(51) Int. Cl. C12Q 1/04; C12Q 1/68; C12Q 1/66; C12M 1/34; C12M 1/40

(52) U.S. Cl. 435/6.1; 435/34; 435/287.1; 435/287.9; 435/39; 435/8; 435/7.1

(57) ABSTRACT

Articles are provided for the detection of cells in a sample. The articles include a release element comprising a cell extractant. The release element includes a shell structure that controls the release of the cell extractant into a liquid mixture containing the sample. Methods of use are also disclosed.

### CROSS REFERENCE TO RELATED APPLICATIONS

This application claims the benefit to U.S. Provisional Patent Application No. 61/175,996, filed May 6, 2009, which is incorporated herein by reference in its entirety.

### BACKGROUND

Various tests are available that can be used to assess the presence of biological analytes in a sample (e.g. surface, water, air, etc). Such tests include those based on the detection of ATP using the firefly luciferase reaction, tests based on the detection of protein using colorimetry, tests based on the detection of microorganisms using microbiological culture techniques, and tests based on detection of microorganisms using immunochemical techniques. Surfaces can be sampled using either a swab device or by direct contact with a culture device such as an agar plate. The sample can be analyzed for the presence of live cells and, in particular, live microorganisms.

Results from these tests are often used to make decisions about the cleanliness of a surface. For example, the test may be used to decide whether food-processing equipment has been cleaned well enough to use for production. Although the above tests are useful in the detection of a contaminated surface, they can require numerous steps to perform the test, they may not be able to distinguish quickly and/or easily the presence of live cells from dead cells and, in some cases, they can require long periods of time (e.g., hours or days) before the results can be determined.

The tests may be used to indicate the presence of live microorganisms. For such tests, a cell extractant is often used to release a biological analyte (e.g., ATP) associated with living cells. The presence of extracellular material (e.g., non-cellular ATP released into the environment from dead or stressed animal cells, plant cells, and/or microorganisms) can create a high “background” level of ATP that can complicate the detection of live cells.

In spite of the availability of a number of methods and devices to detect live cells, there remains a need for a simple, reliable test for detecting live cells and, in particular, live microbial cells.

### SUMMARY

In general, the present disclosure relates to articles and methods for detecting live cells in a sample. The articles and methods make possible the rapid detection (e.g., through fluorescence, chemiluminescence, or a color reaction) of the presence of cells such as bacteria on a surface. In some embodiments, the inventive articles are “sample-ready”, i.e., the articles contain all of the necessary features to detect living cells in a sample. The methods feature the use of a cell extractant to facilitate the release of biological analytes from biological cells. The inventive articles and methods include a release element, which controls the release of an effective amount of cell extractant into a liquid mixture comprising a sample. In some aspects, the inventive articles and methods provide a means to distinguish a biological analyte, such as ATP or an enzyme, that is associated with eukaryotic cells (e.g., plant or animal cells) from a similar or identical biological analyte associated with prokaryotic cells (e.g., bacterial cells). Furthermore, the inventive articles and methods provide a means to distinguish a biological analyte that is free in the environment (i.e., an acellular biological analyte) from a similar or identical biological analyte associated with a living cell.

Thus, in one aspect, the present disclosure provides an article for detecting cells in a sample. The article can comprise a housing with an opening configured to receive a sample acquisition device, a sample acquisition device, and a release element comprising a cell extractant. In some embodiments, the release element can be disposed in the housing. In some embodiments, the release element can be disposed on the sample acquisition device. In some embodiments, the sample acquisition device can further comprise a reagent chamber.

In another aspect, the present disclosure provides an article for detecting cells in a sample. The article can comprise a housing with an opening configured to receive a sample, a sample acquisition device comprising a reagent chamber, a cell extractant, and a release element comprising the cell extractant. The release element can be disposed in the reagent chamber.

In any one of the above embodiments, the article can further comprise a frangible barrier that forms a compartment in the housing. In some embodiments, the frangible barrier can comprise the release element comprising the cell extractant. In some embodiments, the compartment can comprise the release element.

In another aspect, the present disclosure provides an article for detecting cells in a sample. The article can comprise a housing with an opening configured to receive a sample, a release element comprising a cell extractant; a delivery element comprising a detection reagent. In some embodiments, the release element and the delivery element are disposed in the housing.

In any one of the above embodiments, the housing can further comprise a compartment. In any one of the above embodiments, the compartment can further comprise a detection reagent.

In any one of the above embodiments, the detection reagent is selected from the group consisting of an enzyme, an enzyme substrate, an indicator dye, a stain, an antibody, and a polynucleotide.

In another aspect, the present disclosure provides a sample acquisition device with a release element disposed thereon. The release element can comprise a cell extractant. In some embodiments, the cell extractant can comprise a microbial cell extractant. In some embodiments, the cell extractant can comprise a somatic cell extractant.

In another aspect, the present disclosure provides a kit. The kit can comprise a housing with an opening configured to receive a sample, a release element comprising a cell extractant, and a detection system. Optionally, the kit can further comprise a sample acquisition device and the opening in the housing can be configured to receive the sample acquisition device. In some embodiments, the detection system can further comprise a delivery element comprising a detection reagent. In some embodiments, the detection reagent can be selected from the group consisting of an enzyme, an enzyme substrate, an indicator dye, a stain, an antibody, and a polynucleotide.

In another aspect, the present disclosure provides a method of detecting cells in a sample. The method can comprise providing a release element comprising a cell extractant, and a sample suspected of containing cells. The method further can comprise forming a liquid mixture comprising the sample and the release element. The method further can comprise detecting an analyte in the liquid mixture.

In another aspect, the present disclosure provides a method of detecting cells in a sample. The method can comprise providing a sample acquisition device and a housing. The housing can include an opening configured to receive the sample acquisition device and a release element comprising the cell extractant. The release element can be disposed in the housing. The method further can comprise obtaining sample material with the sample acquisition device, forming a liquid mixture comprising the sample material and the release element, and detecting an analyte in the liquid mixture.

In any one of the above embodiments, the release element can comprise an encapsulating agent. In any one of the above embodiments, the encapsulating agent can comprise a core and a shell structure. In any one of the above embodiments, the shell structure can comprise a chromonic material and the core can substantially contain the cell extractant. In some embodiments, the shell structure can be a polymeric shell and the core can substantially contain the cell extractant. In some embodiments, the shell structure can comprise the cell extractant. In any one of the above embodiments, the cell extractant is selected from the group consisting of a quaternary amine, a biguanide, a nonionic surfactant, a cationic surfactant, a phenolic, a cytolytic peptide, and an enzyme.

In any one of the above embodiments, the method further can comprise detecting the analyte using a detection system. In any one of the above embodiments, the method further can comprise quantifying an amount of the analyte. In any one of the above embodiments, the method further can comprise quantifying an amount of the analyte two or more times. In any one of the above embodiments, the method further can comprise releasing the cell extractant from the release element using a release factor.

### GLOSSARY

“Biological analytes”, as used herein, refers to molecules, or derivatives thereof, that occur in or are formed by an organism. For example, a biological analyte can include, but is not limited to, at least one of an amino acid, a nucleic acid, a polypeptide, a protein, a polynucleotide, a lipid, a phospholipid, a saccharide, a polysaccharide, and combinations thereof. Specific examples of biological analytes can include, but are not limited to, a metabolite (e.g., staphylococcal enterotoxin), an allergen (e.g., peanut allergen(s), a hormone, a toxin (e.g., Bacillus diarrheal toxin, aflatoxin, etc.), RNA (e.g., mRNA, total RNA, tRNA, etc.), DNA (e.g., plasmid DNA, plant DNA, etc.), a tagged protein, an antibody, an antigen, and combinations thereof.

“Sample acquisition device” is used herein in the broadest sense and refers to an implement used to collect a liquid, semisolid, or solid sample material. Nonlimiting examples of sample acquisition devices include swabs, wipes, sponges, scoops, spatulas, pipettes, pipette tips, and siphon hoses.

As used herein, “chromonic materials” (or “chromonic compounds”) refers to large, multi-ring molecules typically characterized by the presence of a hydrophobic core surrounded by various hydrophilic groups (see, for example, Attwood, T. K., and Lydon, J. E., Molec. Crystals Liq. Crystals, 108, 349 (1984)). The hydrophobic core can contain aromatic and/or non-aromatic rings. When in solution, these chromonic materials tend to aggregate into a nematic ordering characterized by a long-range order.

As used herein, “release element” refers to a structure that is capable of containing a cell extractant. The release element includes physical and/or chemical components selected to limit the diffusion of a cell extractant from a region of relatively high concentration to a region of relatively low concentration.

“Encapsulating agent” refers to a type of release element. An encapsulating agent, as used herein, is a material that substantially surrounds the cell extractant.

As used herein, “shell structure” refers to a structure or framework forming a type of release element. Generally, the shell structure forms the exterior of the release element.

“Composite release element”, as used herein, refers to a release element that comprises two or more guest molecules.

“Guest molecule”, as used herein, refers to an active compound that facilitates the detection of a biological analyte. Active compounds include cell extractants, binding partners (e.g., antibodies or binding ligands), and detection reagents (e.g., a dye, a stain, an enzyme, an enzyme substrate, a polynucleotide, and the like).

As used herein, the term “hydrogel” refers to a polymeric material that is hydrophilic and that is either swollen or capable of being swollen with a polar solvent. The polymeric material typically swells but does not dissolve when contacted with the polar solvent. That is, the hydrogel is insoluble in the polar solvent. The swollen hydrogel can be dried to remove at least some of the polar solvent.

“Cell extractant”, as used herein, refers to any compound or combination of compounds that alters cell membrane or cell wall permeability or disrupts the integrity of (i.e., lyses or causes the formation of pores in) the membrane and/or cell wall of a cell (e.g., a somatic cell or a microbial cell) to effect extraction or release of a biological analyte normally found in living cells.

“Detection system”, as used herein, refers to the components used to detect a biological analyte and includes enzymes, enzyme substrates, binding partners (e.g. antibodies or receptors), labels, dyes, and instruments for detecting light absorbance or reflectance, fluorescence, and/or luminescence (e.g. bioluminescence or chemiluminescence).

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, a housing that comprises “a” detection reagent can be interpreted to mean that the housing can include “one or more” detection reagents.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The invention will be further explained with reference to the drawing figures listed below, where like structure is referenced by like numerals throughout the several views.

FIG. 1 is a side view of one embodiment of a sample acquisition device with a release element disposed thereon.

FIG. 2 is a partial cross-section view of one embodiment of a sample acquisition device comprising an enclosure containing a release element.

FIG. 3 is a cross-section view of one embodiment of a housing with a release element disposed therein.

FIG. 4 is a cross-section view of the housing of FIG. 3, further comprising a frangible seal.

FIG. 5 is a cross-section view of one embodiment of a housing containing a release element, a frangible seal, and a detection reagent.

FIG. 6A is a cross-section view of one embodiment of a detection device comprising the housing of FIG. 5 and side view of a sample acquisition device disposed in a first position therein.

FIG. 6B is a partial cross-section view of the detection device of FIG. 6A with the sample acquisition device disposed in a second position therein.

FIG. 7 is a partial cross-section view of one embodiment of a detection device comprising a housing, a plurality of frangible seals with a release element disposed there between, and a sample acquisition device.

FIG. 8 is a partial cross-section view of one embodiment of a detection device comprising a housing, a conveyor comprising a release element, and a sample acquisition device.

FIG. 9 is a bottom perspective view of the conveyor of FIG. 8.

FIG. 10 is a cross-sectional view of one embodiment of a release element comprising a shell structure with a core that comprises a guest compound.

FIG. 11 is a cross-sectional view of one embodiment of a release element comprising a shell structure that comprises a guest compound.

FIG. 12 is a cross-sectional view of one embodiment of a release element comprising a shell structure with a core that comprises a first guest compound and a shell structure that comprises a second guest compound.

FIG. 13 is a cross-sectional view of one embodiment of a release element that comprises a plurality of shell structures arranged in successive layers.

FIG. 14 is a cross-sectional view of one embodiment of a release element that comprises a plurality of shell structures arranged separately within an outer shell structure.

### DETAILED DESCRIPTION

All patents, patent applications, government publications, government regulations, and literature references cited in this specification are hereby incorporated herein by reference in their entirety. In case of conflict, the present description, including definitions, will control.

Biological analytes can be used to detect the presence of biological material, such as live cells in a sample. Biological analytes can be detected by various reactions (e.g., binding reactions, catalytic reactions, and the like) in which they can participate.

Chemiluminescent reactions can be used in various forms to detect cells, such as bacterial cells, in fluids and in processed materials. In some embodiments of the present disclosure, a chemiluminescent reaction based on the reaction of adenosine triphosphate (ATP) with luciferin in the presence of the enzyme luciferase to produce light provides the chemical basis for the generation of a signal to detect a biological analyte, ATP. Since ATP is present in all living cells, including all microbial cells, this method can provide a rapid assay to obtain a quantitative or semiquantitative estimate of the number of living cells in a sample. Early discourses on the nature of the underlying reaction, the history of its discovery, and its general area of applicability, are provided by E. N. Harvey (1957), A History of Luminescence: From the Earliest Times Until 1900, Amer. Phil. Soc., Philadelphia, Pa.; and W. D. McElroy and B. L. Strehler (1949), Arch. Biochem. Biophys. 22:420-433.

ATP detection is a reliable means to detect bacteria and other microbial species because all such species contain some ATP. Chemical bond energy from ATP is utilized in the bioluminescent reaction that occurs in the tails of the firefly Photinus pyralis. The biochemical components of this reaction can be isolated free of ATP and subsequently used to detect ATP in other sources. The mechanism of this firefly bioluminescence reaction has been well characterized (DeLuca, M., et al., 1979 Anal. Biochem. 95:194-198).

The inventive articles and methods of the present disclosure provide simple means for conveniently controlling the release of biological analytes from living cells in order to determine the presence, optionally the type (e.g., microbial or nonmicrobial), and optionally the quantity of living cells in an unknown sample. The articles and methods include a release element comprising a cell extractant.

### Release Element:

Release elements according to the present disclosure include an encapsulating material that holds and/or comprises a cell extractant. The encapsulating materials can act as a physical barrier and/or a diffusion barrier to prevent the immediate dissolution and/or dispersion, for a period of time, of an effective amount of the cell extractant into a liquid mixture (for example, an aqueous mixture comprising a sample).

In some embodiments, the encapsulating materials can comprise a matrix material. Release elements comprising an encapsulating material that includes a matrix material are disclosed in U.S. Patent Application No. 61/175,980, filed May 6, 2009 and entitled “ARTICLES WITH MATRIX COMPRISING A CELL EXTRACTANT AND BIODETECTION METHODS THEREOF”, which is incorporated herein by reference in its entirety.

In some embodiments, the encapsulating materials may be activated to release an effective amount of cell extractant after the encapsulant is exposed to activating stimuli such as pressure, shear, heat, light, pH change, exposure to another chemical, ionic strength change and the like. Activation may result in, for example, dissolution or partial dissolution of the encapsulating material, permeabilization of the encapsulating material (e.g. disruption of a lipid bilayer), and/or disintegration or partial disintegration of the encapsulating material (e.g., by fracturing or melting a solid material such as, for example microcrystalline wax).

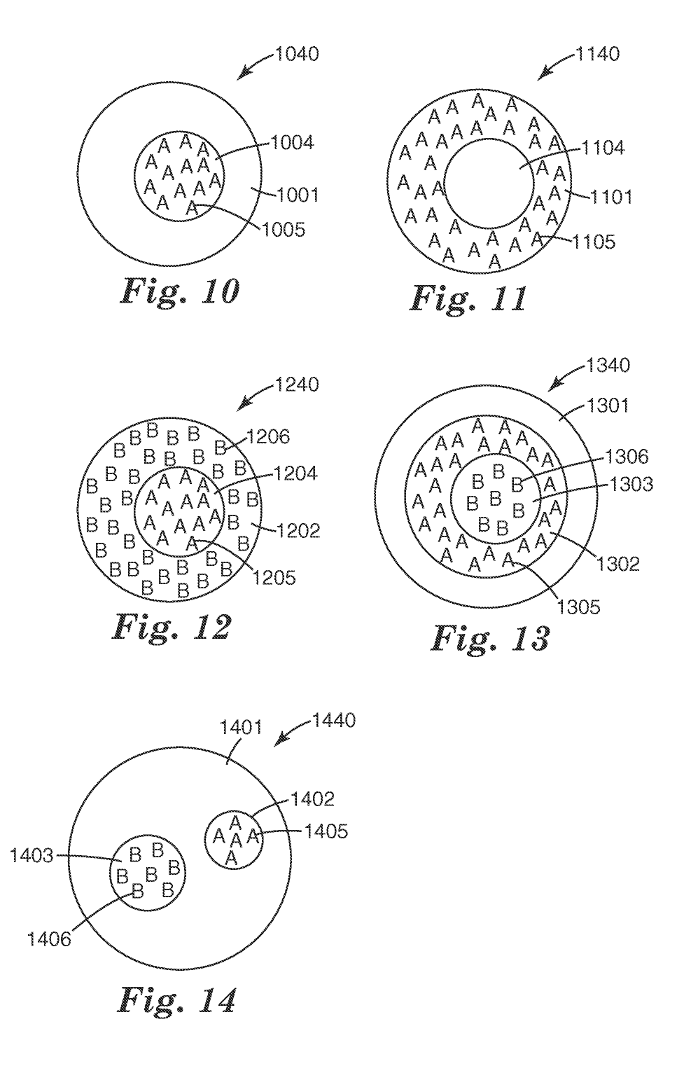

In some embodiments, the release element forms a shell structure. FIG. 10 shows an embodiment of a release element 1040 according to the present disclosure. Release element 1040 includes a shell structure 1001 and a core 1004. Located in the core 1004 is guest compound 1005 (e.g., a cell extractant as described herein). The core 1004 can comprise a liquid, a solid, a semisolid or combinations thereof. Guest compound 1005 may be dissolved and/or dispersed therein.

In some embodiments, the shell structure of a release element can comprise a cell extractant. FIG. 11 shows an embodiment of a release element 1140 according to the present disclosure. Release element 1140 includes a shell structure 1101 comprising a guest compound 1105 (e.g., a cell extractant as described herein). Release element 1140 further comprises a core 1104, which can comprise a liquid, a solid, a semisolid, or combinations thereof.

In some embodiments, a release element can comprise two or more guest molecules. FIG. 12 shows an embodiment of a composite release element 1240 according to the present disclosure. Composite release element 1240 includes a shell structure 1202 comprising a first guest molecule 1206 and a core 1204 comprising a second guest molecule 1205. In some embodiments, first and second guest compounds (1204 and 1205, respectively) can be the same compound. In some embodiments, first and second guest compounds (1206 and 1205, respectively) can be different compounds. In some embodiments, a least one guest compound can be a cell extractant. In some embodiments, the at least one guest compound can be a detection reagent as described herein. In some embodiments, at least one guest compound can be a cell extractant and at least one guest compound can be a detection reagent.

In some embodiments, a release element can comprise two or more shell structures. FIG. 13 shows an embodiment of a release element 1340 comprising a first shell structure 1301, a second shell structure 1302, and a third shell structure 1303 (the core of release element 1340 is not shown in this view). In this embodiment, second shell structure 1302 comprises first guest compound 1305 and third shell structure 1303 comprises second guest compound 1306. In this embodiment, each successive shell structure is encapsulated by the next shell structure, in an onion-like fashion. In some embodiments, first and second guest compounds (1305 and 1306, respectively) can be the same compound. In some embodiments, first and second guest compounds (1305 and 1306, respectively) can be different compounds. In some embodiments, a least one guest compound can be a cell extractant. In some embodiments, the at least one guest compound can be a detection reagent as described herein. In some embodiments, at least one guest compound can be a cell extractant and at least one guest compound can be a detection reagent.

FIG. 14 shows an alternative embodiments of a release element 1440 comprising two or more shell structures. Release element 1440 includes a first shell structure 1401, a second shell structure 1402, and a third shell structure 1403. The second shell structure 1402 comprises a first guest compound 1405 and the third shell structure comprises a second guest compound 1406. In this embodiment, both the second shell structure 1402 and third shell structure 1403 are encapsulated by the first shell structure 1401. In some embodiments, first and second guest compounds (1405 and 1406, respectively) can be the same compound. In some embodiments, first and second guest compounds (1405 and 1406, respectively) can be different compounds. In some embodiments, a least one guest compound can be a cell extractant. In some embodiments, the at least one guest compound can be a detection reagent as described herein. In some embodiments, at least one guest compound can be a cell extractant and at least one guest compound can be a detection reagent.

Suitable shell structures of the present disclosure include polymer shells comprising, for example, cellulose, cellulose derivatives (e.g., cellulose ethers, cellulose esters, cellulose nitrate, cellulose triacetate, cellulose acetate phthalate, methyl cellulose, ethyl cellulose, hydroxypropyl cellulose, hydroxypropyl methylcellulose, carboxymethyl cellulose, and hydroxypropyl methylcellulose phthalate), polymers or copolymers of acrylates or copolymers of acrylate derivatives (e.g., polyacrylates, polymethylacrylates, poly(acrylate-methacrylate), poly(methacrylate-methylmehacrylate), polyethylacrylate-methylmethacrylate, poly(ethylacrylate-methylmethacrylate-trimethylammonioethylmethacrylate chloride), and poly (ethylacrylate-methylmethacrylate-trimethylammonioethylmethacrylate chloride)), proteins (e.g., albumin, gelatin, zein, casein, collagen, and fibrinogen), vinyl polymers (e.g., polyvinyl chloride, polyvinyl acetate, polyvinyl alcohol, polystyrene, and polyacrylonitrile), gums (e.g., guar gum, gum arabic, xanthan gum, locust bean gum), natural or modified starches, dextrins, dextrans, chitosan, alginates, and a combination of any two or more of the foregoing, as described in U.S. Pat. No. 7,485,609, which is incorporated herein by reference in its entirety.

Additionally or alternatively, suitable shell structures of the present disclosure include shell structures comprising a chromonic material. Suitable chromonic materials include those that form shell layers as described in, for example, U.S. Patent Application Publication No. US 2007/0148458, which is incorporated herein by reference in its entirety.

The shell layers comprised of chromonic materials can be particularly useful for the encapsulation and controlled release of guest compounds (for example, cell extractants). For example, a cell extractant can be encapsulated in a chromonic nanoparticle as described in U.S. Patent Application Publication No. US 2007/0148458. The chromonics can protect the cell extractant from certain environmental conditions and then controllably deliver the cell extractant under other environmental conditions. The shell comprising a complex comprising chromonic material, multivalent cations, and acid anions selected from the group consisting of HCO₃⁻, PO₄³⁻, CH₃CHOHCOO⁻, C₃H₅O(COO)₃³⁻, BO₃³⁻, and SO₄²⁻ (the “complexed shell”), however, can provide increased protection from certain environmental conditions as compared to chromonics alone.

Multilayered chromonic structures comprising a chromonic nanoparticle encapsulated in one or more shell layers of chromonic material are known in the art. A cell extractant can be encapsulated in the chromonic nanoparticle and/or in one or more chromonic shell layers that encapsulate the nanoparticle. The complexed shell can be used in combination with one or more chromonic shell layers (for example, the complexed shell could be the innermost shell layer, an intermediate shell layer, the outermost shell layer, or any combination thereof). Complexed shell layers can provide increased flexibility for the controlled release of cell extractants (e.g., sustained delivery).

Shell layers according to the present disclosure also include wax structures (e.g., capsules) that substantially surround the cell extractant. In some embodiments, a generally unitary body (e.g. a liquid, a solid, a plurality of solids such as particles, a semisolid, or combinations thereof) of cell extractant can be disposed inside an outer wax shell. As the wax disintegrates (e.g., by thermal melting or mechanical disruption), the cell extractant is released from the wax. Suitable waxes include paraffin wax, microcrystalline wax, and derivatives and/or combinations thereof.

Shell layers according to the present disclosure also include lipid bilayers (e.g., liposomes). The liposomes can be formed by liposome-forming techniques known in the art. The liposomes can be formed out of a solution containing a cell extractant such that at least a portion of the cell extractant is trapped in the core of the liposome. In contrast to other types of release elements, which can comprise cell extractants that may disrupt any type of lipid bilayer, liposome release elements can contain cell extractants that do not substantially impair the integrity of the lipid bilayers of the liposome. Non-limiting examples of such cell extractants include polypeptides, such as lysozyme and lysostaphin, and antibiotics that do not substantially impair the liposomes.

When the liposome is contacted with a liquid (e.g., an aqueous liquid containing a sample), the cell extractant may be released from the liposome by, for example, diffusion through the lipid bilayer. In some embodiments, the entire contents (e.g., the cell extractant solution) of the liposome may be released in a “burst” with a release factor that disrupts the integrity of the lipid bilayer using thermal or mechanical energy (e.g., heat, freeze-thaw, or sonication) or by adding a chemical release factor to permeabilize and/or solublize the lipid bilayer. In some embodiments, disruption of liposomes can be triggered using cytolytic peptides, as described in U.S. Patent Application No. 61/028,896 (Attorney Docket No. 63973US002) filed on Feb. 14, 2008 and entitled “POLYPEPTIDES FOR MICROBIAL DETECTION”, which is incorporated herein by reference in its entirety.

In some embodiments, release elements of the present disclosure can include shell structures comprising a cell extractant coated on a substrate. Release elements comprising coated substrates are described in U.S. Patent Application No. 61/175,987, filed May 6, 2009 and entitled “COATED SUBSTRATES COMPRISING A CELL EXTRACTANT AND BIODETECTION METHODS THEREOF”, which is incorporated herein by reference in its entirety.

### Cell Extractants:

In some embodiments, chemical cell extractants include biochemicals, such as proteins (e.g., cytolytic peptides and enzymes). In some embodiments, the cell extractant increases the permeability of the cell, causing the release of biological analytes from the interior of the cell. In some embodiments, the cell extractant can cause or facilitate the lysis (e.g., rupture or partial rupture) of a cell.

In some embodiments, cell extractants include chemicals and mixtures of chemicals that are known in the art and include, for example, surfactants and quaternary amines, biguanides, surfactants, phenolics, cytolytic peptides, and enzymes. Typically, the cell extractant is not avidly bound (either covalently or noncovalently) to the release element and can be released from the release element when the release element is contacted with a liquid (e.g., an aqueous liquid comprising a sample).

Surfactants generally contain both a hydrophilic group and a hydrophobic group. The release element may contain one or more surfactants selected from anionic, nonionic, cationic, ampholytic, amphoteric and zwitterionic surfactants and mixtures thereof. A surfactant that dissociates in water and releases cation and anion is termed ionic. When present, ampholytic, amphoteric and zwitterionic surfactants are generally used in combination with one or more anionic and/or nonionic surfactants. Nonlimiting examples of suitable surfactants and quaternary amines include TRITON X-100, Nonidet P-40 (NP-40), Tergitol, Sarkosyl, Tween, SDS, Igepal, Saponin, CHAPSO, benzalkonium chloride, benzethonium chloride, ‘cetrimide’ (a mixture of dodecyl-, tetradecyl- and hexadecyl-trimethylammoium bromide), cetylpyridium chloride, (meth)acrylamidoalkyltrimethylammonium salts (e.g., 3-methacrylamidopropyltrimethylammonium chloride and 3-acrylamidopropyltrimethylammonium chloride) and (meth)acryloxyalkyltrimethylammonium salts (e.g., 2-acryloxyethyltrimethylammonium chloride, 2-methacryloxyethyltrimethylammonium chloride, 3-methacryloxy-2-hydroxypropyltrimethylammonium chloride, 3-acryloxy-2-hydroxypropyltrimethylammonium chloride, and 2-acryloxyethyltrimethylammonium methyl sulfate). Other suitable monomeric quaternary amino salts include a dimethylalkylammonium group with the alkyl group having 2 to 22 carbon atoms or 2 to 20 carbon atoms. That is, the monomer includes a group of formula —N(CH₃)₂(CₙH₂₊₁)⁺ where n is an integer having a value of 2 to 22. Exemplary monomers include, but are not limited to monomers of the following formula

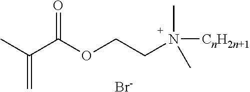

where n is an integer in the range of 2 to 22.

Non-limiting examples of suitable biguanides, which include bis-biguanides, include polyhexamethylene biguanide hydrochloride, p-chlorophenyl biguanide, 4-chloro-benzhydryl biguanide, alexidine, halogenated hexidine such as, but not limited to, chlorhexidine (1,1′-hexamethylene-bis-5-(4-chlorophenyl biguanide), and salts thereof.

Non-limiting examples of suitable phenolics include phenol, salicylic acid, 2-phenylphenol, 4-t-amylphenol, Chloroxylenol, Hexachlorophene, 4-chloro-3,5-dimethylphenol (PCMX), 2-benzyl-4-chlorophenol, triclosan, butylated hydroxytoluene, 2-Isopropyl-5-methyl phenol, 4-Nonylphenol, xylenol, bisphenol A, Orthophenyl phenol, and Phenothiazines, such as chlorpromazine, prochlorperazine and thioridizine.

Non-limiting examples of suitable cytolytic peptides include A-23187 (Calcium ionophore), Dermaseptin, Listerolysin, Ranalexin, Aerolysin, Dermatoxin, Maculatin, Ranateurin, Amphotericin B, Direct lytic factors from animal venoms, Magainin, Rugosin, Ascaphin, Diptheria toxin, Maxymin, Saponin, Aspergillus haemolysin, Distinctin, Melittin, Staphylococcus aureus toxins, (α, β, χ, [symbol] ), Alamethicin, Esculetin, Metridiolysin, Streptolysin O, Apolipoproteins, Filipin, Nigericin, Streptolysin S, ATP Translocase, Gaegurin, Nystatin, Synexin, Bombinin, GALA, Ocellatin, Surfactin, Brevinin, Gramicidin, P25, Tubulin, Buforin, Helical erythrocyte lysing peptide, Palustrin, Valinomycin, Caerin, Hemolysins, Phospholipases, Vibriolysin, Cereolysin, Ionomycin, Phylloxin, Colicins, KALA, Polyene Antibiotics, Dermadistinctin, LAGA, Polymyxin B.

Non-limiting examples of suitable enzymes include lysozyme, lysostaphin, bacteriophage lysins, achromopeptidase, labiase, mutanolysin, streptolysin, tetanolysin, a-hemolysin, lyticase, lysing enzymes from fungi, cellulase, pectinase, Driselase®Viscozyme® L, pectolyase.

Any other known cell extractants that are compatible with the release element can be used. These include, but are not limited to, chlorhexidine salts such as chlorhexidine gluconate (CHG), parachlorometaxylenol (PCMX), triclosan, hexachlorophene, fatty acid monoesters and monoethers of glycerin and propylene glycol such as glycerol monolaurate, Cetyl Trimethylammonium Bromide (CTAB), glycerol monocaprylate, glycerol monocaprate, propylene glycol monolaurate, propylene glycol monocaprylate, propylene glycol moncaprate, phenols, surfactants and polymers that include a (C12-C22) hydrophobe and a quaternary ammonium group or a protonated tertiary amino group, quaternary amino-containing compounds such as quaternary silanes and polyquaternary amines such as polyhexamethylene biguanide, transition metal ions such as copper containing compounds, zinc containing compounds, and silver containing compounds such as silver metal, silver salts such as silver chloride, silver oxide and silver sulfadiazine, methyl parabens, ethyl parabens, propyl parabens, butyl parabens, octenidene, 2-bromo-2-nitropropane-1,3 diol, or mixtures of any two or more of the foregoing.

Suitable cell extractants also include dialkyl ammonium salts, including N-(n-dodecyl)-diethanolamine; cationic ethoxylated amines, including ‘Genaminox K-10’, Genaminox K-12, ‘Genamin TCL030’, and ‘Genamin C100’; amidines, including propamidine and dibromopropamidine; peptide antibiotics, including polymyxin B and nisin; polyene antibiotics, including nystatin, amphotericin B, and natamycin; imidazoles, including econazole, clotramizole and miconazole; oxidizing agents, including stabilized forms of chlorine and iodine; and the cell extractants described in U.S. Pat. No. 7,422,868, which is incorporated herein by reference in its entirety.

Cell extractants are preferably chosen not to inactivate the detection system (e.g., a detection reagent such as luciferase enzyme) of the present invention. For microbes requiring harsher cell extractants (e.g., ionic detergents etc.), modified detection systems (such as luciferases exhibiting enhanced stability in the presence of these agents, such as those disclosed in U.S. Patent Application Publication No. 2003/0104507, which is hereby incorporated by reference in its entirety) are particularly preferred.

Methods of the present invention provide for the release of an effective amount of cell extractant from a release element to cause the release of biological analytes from a live cell. The present disclosure includes a variety of cell extractants known in the art and each of which may be released from the release element at a different rate and may exert its effect on living cells at a different concentration than the others. The following will provide guidance concerning the factors to be considered in selecting the cell extractant and the in determining an effective amount to include in the release element.

It is known in the art that the efficacy of any cell extractant is determined primarily by two factors—concentration and exposure time. That is, in general, the higher the concentration of a cell extractant, the greater the effect (e.g., permeabilization of the cell membrane and/or release of biological analytes from the cell) it will have on a living cell. Also, at any given concentration of cell extractant, in general, the longer you expose a living cell to the cell extractant, the greater the effect of the cell extractant. Other extrinsic factors such as, for example, pH, co-solvents, ionic strength, and temperature are known in the art to affect the efficacy of certain cell extractant. It is known that these extrinsic factors can be controlled by, for example, temperature controllers, buffers, sample preparation, and the like. These factors, as well as the cell extractant, can also have effects on the detection systems used to detect biological analytes. It is well within the grasp of a person of ordinary skill to perform a few simple experiments to determine an effective amount of cell extractant to produce the articles and perform the methods of the present disclosure. Further guidance is provided in the Examples described herein.

Initial experiments to determine the effect of various concentrations of the cell extractant on the cells and/or the detection system can be performed. Initially, a putative release element comprising a cell extractant can be screened for its effect on the biological analyte detection system. For example, the release element comprising a cell extractant can be placed into an ATP assay (without bacterial cells) similar to the ATP assay described in Example 4. The assay can be run with solutions of reagent-grade ATP (e.g. from about 0.1 to about 100 picomoles of ATP) and the amount of bioluminescence emitted by the luciferase reaction in the sample with the release element comprising a cell extractant can be compared to the amount of bioluminescence emitted by a sample without the release element comprising a cell extractant. Preferably, the amount of bioluminescence in the sample with the release element comprising a cell extractant is greater than 50% of the amount of bioluminescence in the sample without the release element comprising a cell extractant. More preferably, the amount of bioluminescence in the sample with the release element comprising a cell extractant is greater than 90% of the amount of bioluminescence in the sample without the release element comprising a cell extractant. Most preferably, the amount of bioluminescence in the sample with the release element comprising a cell extractant is greater than 95% of the amount bioluminescence in the sample without the release element comprising a cell extractant.

Additionally, the effect of the cell extractant on the release of the biological analyte from the cells can be determined experimentally, as described in Example 3 below. For example, liquid suspensions of cells (e.g., microbial cells such as Staphylococcus aureus) are exposed to relatively broad range of concentrations of a cell extractant (e.g., BARDAC 205M) for a period of time (e.g. up to several minutes) in the presence of a detection system to detect biological analytes from a cell (e.g., an ATP detection system comprising luciferin, luciferase, and a buffer at about pH 7.6 to 7.8). The biological analyte is measured periodically, with the first measurement usually performed immediately after the cell extractant is added to the mixture, to determine whether the release of the biological analyte (in this example, ATP) from the cells can be detected. The results can indicate the optimal conditions (i.e., liquid concentration of cell extractant and exposure time) to detect the biological analyte released from the cells. The results may also indicate that, at higher concentrations of cell extractant, the cell extractant may be less effective in releasing the biological analyte (e.g., ATP) and/or may interfere with the detection system (i.e., may absorb the light or color generated by the detection reagents).

After the effective amount of cell extractant in liquid mixtures is determined, consideration should be given to the amount of cell extractant to incorporate into the release element by the methods described herein. When the release element comprising a cell extractant forms a liquid mixture (e.g., a sample suspected of containing live cells in an aqueous suspension) the cell extractant diffuses out of the release element until a concentration equilibrium of the cell extractant, between the release element and the liquid, is reached. Without being bound by theory, it can be assumed that, until the equilibrium is reached, a concentration gradient of cell extractant will exist in the liquid, with a higher concentration of extractant present in the portion of the liquid proximal the release element. When the concentration of the cell extractant reaches an effective concentration in a portion of the liquid containing a cell, the cell releases biological analytes. The released biological analytes are thereby available for detection by a detection system.

Achieving an effective concentration of cell extractant in the liquid containing the sample can be controlled by several factors. For example, the amount of cell extractant loaded into the release element can affect final concentration of cell extractant in the liquid at equilibrium. Additionally, the amount of release element and, in some embodiments, the amount of surface area of the release element in the liquid mixture can affect the rate of release of the cell extractant from the release element and the final concentration of cell extractant in the liquid at equilibrium. Furthermore, the temperature of the liquid can affect the rate at which the release element releases the cell extractant. Other factors, such as the ionic properties and or hydrophobic properties of the cell extractant and the release element may affect the amount of cell extractant released from the release element and the rate at which the cell extractant is released from the release element. All of these factors can be optimized with routine experimentation by a person of ordinary skill to achieve the desired parameters (e.g., manufacturing considerations for the articles and the time-to-result for the methods) for detection of cells in a sample. In general, it is desirable to incorporate at least enough cell extractant into the release element to achieve the effective amount (determined by the experimentation using the cell extractant without a release element) when the cell extractant reaches equilibrium between the release element and the volume of liquid comprising the sample material. It may be desirable to add a larger amount of cell extractant to the release element (than the amount determined by experimentation using the cell extractant without a release element) to reduce the amount of time it take for the release element to release an effective amount of cell extractant.

The release element can be contacted with the liquid sample material either statically, dynamically (i.e., with mixing by vibration, stirring, aeration or compressing, for example), or a combination thereof. In certain embodiments wherein the release element comprises a frangible shell comprising and/or containing a cell extractant, compressing the release element (e.g., pressing the composition against a surface and/or crushing the composition) can cause a faster release of an effective amount of cell extractant. Compressing the release element can include, for example, pressing the composition against a surface and/or crushing the composition. Thus, in some embodiments, mixing can advantageously provide a faster release of cell extractant and thereby a faster detection of biological analytes (e.g., from live cells) in a sample. In some embodiments, compressing the release element (e.g., by exerting pressure against the composition using a sample acquisition device such as a swab or a spatula, a conveyor (described below) or some other suitable implement) can advantageously provide a faster release of cell extractant and thereby a faster detection of biological analytes in a sample. Additionally, the step of compressing the release element can be performed to accelerate the release of the cell extractant at a time that is convenient for the operator. In some embodiments, static contact can delay the release of an effective amount of cell extractant and thereby provide additional time for the operator to carry out other procedures (e.g., reagent additions, instrument calibration, and/or specimen transport) before detecting the biological analytes. In some embodiments, it may be advantageous to hold the mixture statically until a first biological analyte measurement is taken and then dynamically mix the sample to reduce the time necessary to release an effective amount of cell extractant.

It is fully anticipated that the most preferred concentration(s) or concentration range(s) functional in the methods of the invention will vary for different microbes and for different cell extractants and may be empirically determined using the methods described herein or commonly known to those skilled in the art.

### Samples and Sample Acquisition Devices:

Articles and methods of the present disclosure provide for the detection of biological analytes in a sample. In some embodiments, the articles and methods provide for the detection of biological analytes from live cells in a sample. In certain preferred embodiments, the articles and methods provide for the detection of live microbial cells in a sample. In certain preferred embodiments, the articles and methods provide for the detection of live bacterial cells in a sample.

The term “sample” as used herein, is used in its broadest sense. A sample is a composition suspected of containing a biological analyte (e.g., ATP) that is analyzed using the invention. While often a sample is known to contain or suspected of containing a cell or a population of cells, optionally in a growth media, or a cell lysate, a sample may also be a solid surface, (e.g., a swab, membrane, filter, particle), suspected of containing an attached cell or population of cells. It is contemplated that for such a solid sample, an aqueous sample is made by contacting the solid with a liquid (e.g., an aqueous solution) which can be mixed with release elements of the present disclosure. Filtration of the sample is desirable in some cases to generate a sample, e.g., in testing a liquid or gaseous sample by a process of the invention. Filtration is preferred when a sample is taken from a large volume of a dilute gas or liquid. The filtrate can be contacted with hydrogels of the present disclosure, for example after the filtrate has been suspended in a liquid.

Suitable samples include samples of solid materials (e.g., particulates, filters), semisolid materials (e.g., a gel, a liquid suspension of solids, or a slurry), a liquid, or combinations thereof. Suitable samples further include surface residues comprising solids, liquids, or combinations thereof. Non-limiting examples of surface residues include residues from environmental surfaces (e.g., floors, walls, ceilings, fomites, equipment, water, and water containers, air filters), food surfaces (e.g., vegetable, fruit, and meat surfaces), food processing surfaces (e.g., food processing equipment and cutting boards), and clinical surfaces (e.g., tissue samples, skin and mucous membranes).

The collection of sample materials, including surface residues, for the detection of biological analytes is known in the art. Various sample acquisition devices, including spatulas, sponges, swabs and the like have been described. The present disclosure provides sample acquisition devices with unique features and utility, as described herein.

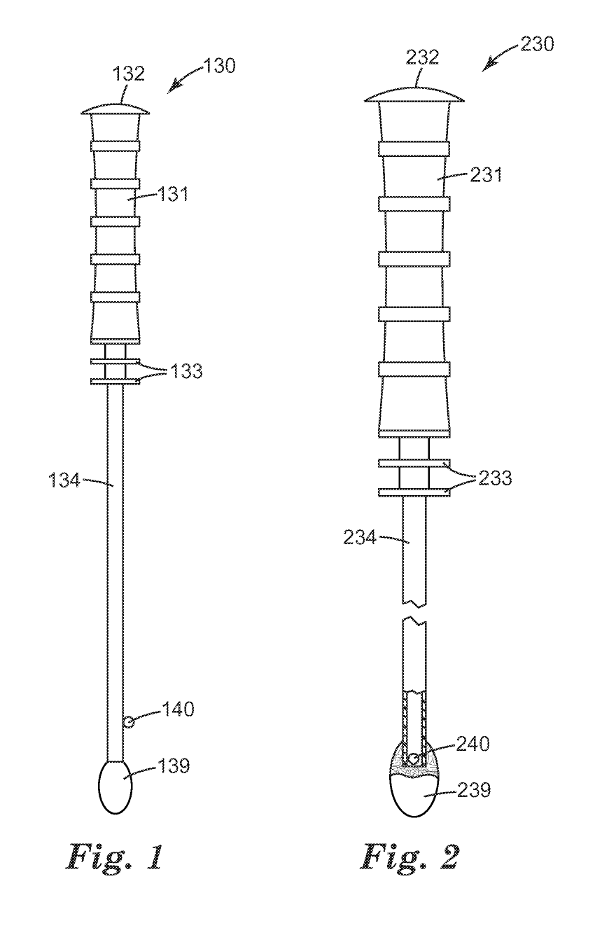

Turning now to the Figures, FIG. 1 shows a side view of one embodiment of a sample acquisition device 130 according to the present disclosure. The sample acquisition device 130 comprises a handle 131 which can be grasped by the operator while collecting a sample. The handle comprises an end 132 and, optionally, a plurality of securing members 133. Securing members 133 can be proportioned to slideably fit into a housing (such as housing 320 or housing 420 shown in FIGS. 3 and 4, for example). In some embodiments, the securing members 133 can form a liquid-resistant seal to resist the leakage of fluids from a housing.

The sample acquisition device 130 further comprises an elongated shaft 134 and a tip 139. In some embodiments, the shaft 134 can be hollow. The shaft 134 comprises a tip 139, positioned near the end of the shaft 134 opposite the handle 131. The tip 139 can be used to collect sample material and can be constructed from porous materials, such as fibers (e.g., rayon or Dacron fibers) or foams (e.g., polyurethane foam) which can be affixed to the shaft 134. In some embodiments, the tip 139 can be a molded tip as described in U.S. Patent Application No. 61/029,063, filed Dec. 5, 2007 and entitled, “SAMPLE ACQUISITION DEVICE”, which is incorporated herein by reference in its entirety. The construction of sample acquisition devices 130 is known in the art and can be found, for example, in U.S. Pat. No. 5,266,266, which is incorporated herein by reference in their entirety.

Optionally, the sample acquisition device 130 can further comprise a release element 140 (shown as a spherical structure) comprising a cell extractant. In some embodiments, the release element 140 is positioned in or on the sample acquisition device 130 at a location other than the tip 139 that is used to collect the sample (e.g., on the shaft 134, as shown in FIG. 1). In some embodiments, the release element 140 can be positioned on an exterior surface of the sample acquisition device 130 (as shown in FIG. 1). In some embodiments, the release element 140 can be positioned on an interior surface of the sample acquisition device 130 (as shown in FIG. 2). The release element 140 can be coated onto shaft 134 as described herein or it can be adhered to the shaft 134 by, for example, a pressure-sensitive adhesive or a water-soluble adhesive (not shown). The adhesive should be selected for its compatibility with the detection system used to detect a biological analyte from live cells (i.e., the adhesive should not significantly impair the accuracy or sensitivity of the detection system).

In use, the tip 139 of a sample acquisition device 130 is contacted with a sample material (e.g., a solid, a semisolid, a liquid suspension, a slurry, a liquid, a surface, and the like) to obtain a sample suspected of containing cells. The sample acquisition device 130 can be used to transfer the sample to a detection system as described herein.

FIG. 2 shows a partial cross-sectional view of another embodiment of a sample acquisition device 230 according to the present disclosure. In this embodiment, the sample acquisition device 230 comprises a handle 231 with an end 232, optional securing members 233 to slideably fit within a housing (not shown), a hollow elongated shaft 234, and a tip 239 comprising porous material. The sample acquisition device 230 further comprises a release element 240 (shown as a spherical structure), which comprises a cell extractant, disposed in the interior portion of the shaft 234. Thus, the sample acquisition device 230 provides an enclosure (shaft 234) containing the release element 240. The material comprising the tip 239 is porous enough to permit liquids to flow freely into the interior of the shaft 234 without permitting the release element 240 to pass through the material and out of the tip 239.

In use, sample acquisition device 230 can be used to contact surfaces, preferably dry surfaces, to obtain sample material. After the sample is obtained, the tip 239 of the sample acquisition device 230 is moistened with a liquid (e.g. water or a buffer; optionally, including a detection reagent such as an enzyme and/or an enzyme substrate), thereby permitting an effective amount of the cell extractant to be released from the release element 240 and to contact the sample material. The release of an effective amount of cell extractant from release element 240 permits the sample acquisition device 230 to be used in methods to detect biological analytes from live cells as described herein.

Another embodiment (not shown) of a sample acquisition device including a release element comprising a cell extractant can be derived from the “Specimen Test Unit” disclosed by Nason in U.S. Pat. No. 5,266,266 (hereinafter, referred to as the “Nason patent”). In particular, referring to FIGS. 7-9 of the Nason patent, the handle of the sample acquisition devices described herein can be modified to embody Nason's functional elements of the housing base 14 (which forms reagent chamber 36) and the seal fitting 48, which includes central dispense passage 50 (optional, with housing cap 30) connected to the hollow swab shaft 22. The central passage 50 of the seal fitting 48 can be closed by a break-off nib 52 in the form of an extended rod segment 54 connected to the seal fitting 48 at the inboard end of the passage 50 via a reduced diameter score 56. Thus, in one embodiment of the present disclosure, the sample acquisition device handle comprises a reagent chamber, as described by Nason. The reagent chamber located in the handle of the sample acquisition device of this embodiment includes release element particles (e.g., shell structures) comprising a cell extractant. Thus, the sample acquisition device of this embodiment provides an enclosure (reagent chamber 36) containing the release element. In this embodiment, the release element particles are not suspended in a liquid medium that causes the release of the cell extractant from the composition. The release element particles are proportioned and shaped to allow free passage of the individual particles into and through the central passage 50 and the hollow shaft 22.

In use, the sample acquisition device comprising a handle including a reagent chamber can be used to obtain a sample as described herein. If the sample is a liquid, the break-off nib 52 can be actuated, as described in the Nason patent, enabling the passage of the release element through the shaft to contact the liquid sample in the swab tip, thereby forming a liquid mixture comprising the sample and the composition. The liquid mixture comprising the sample and the release element can be used for the detection of a biological analyte associated with a live cell, as described herein. If the sample is a solid or semi-solid, the tip of the sample acquisition device can be contacted or submersed in a liquid solution and the break-off nib 52 can be actuated, as described in the Nason patent, enabling the passage of the release element through the shaft to contact the liquid sample in the swab tip, thereby forming a liquid mixture comprising the sample and the composition. The liquid mixture comprising the sample and the release element can be used for the detection of a biological analyte associated with a live cell, as described herein.

### Detection Devices:

FIG. 3 shows a cross-sectional view of one embodiment of a housing 320 of a detection device according to the present disclosure. The housing 320 comprises an opening 322 configured to receive a sample acquisition device and at least one wall 324. Disposed in the housing 320 is a release element 340 comprising a cell extractant. Thus, the housing 320 provides an enclosure containing the release element 340.

In FIG. 3, the release element 340 is shown in the form of a generally spherical shell structure. It will be appreciated that a spherical shell structure is just one example of a variety of shaped release elements that are suitable for use in housing 320.

It should be recognized that in this and all other embodiments (for example, the illustrated embodiments of FIGS. 1, 2, 4, 5, 6A-B, 7, and 8), the release element (e.g., release element 340) may include a plurality (for example, at least 2, 3, 4, 5, up to 10, up to 20, up to 50, up to 100, up to 500, up to 1000) of shell structures. For example, release element 340 can comprise up to 2, up to 3, up to 4, up to 5, up to 10, up to 20, up to 50, up to 100, up to 500, up to 1000 or more shell structures.

The wall 324 of the housing 320 can be cylindrical, for example. It will be appreciated that other useful geometries, some including a plurality of walls 324, are possible and within the grasp of one of ordinary skill in the appropriate art. The housing 320 can be constructed from a variety of materials such as plastic (e.g., polypropylene, polyethylene, polycarbonate) or glass. Preferably, at least a portion of the housing 320 is constructed from materials that have optical properties that allow the transmission of light (e.g., visible light). Suitable materials are well known in devices used for biochemical assays such as ATP tests, for example.

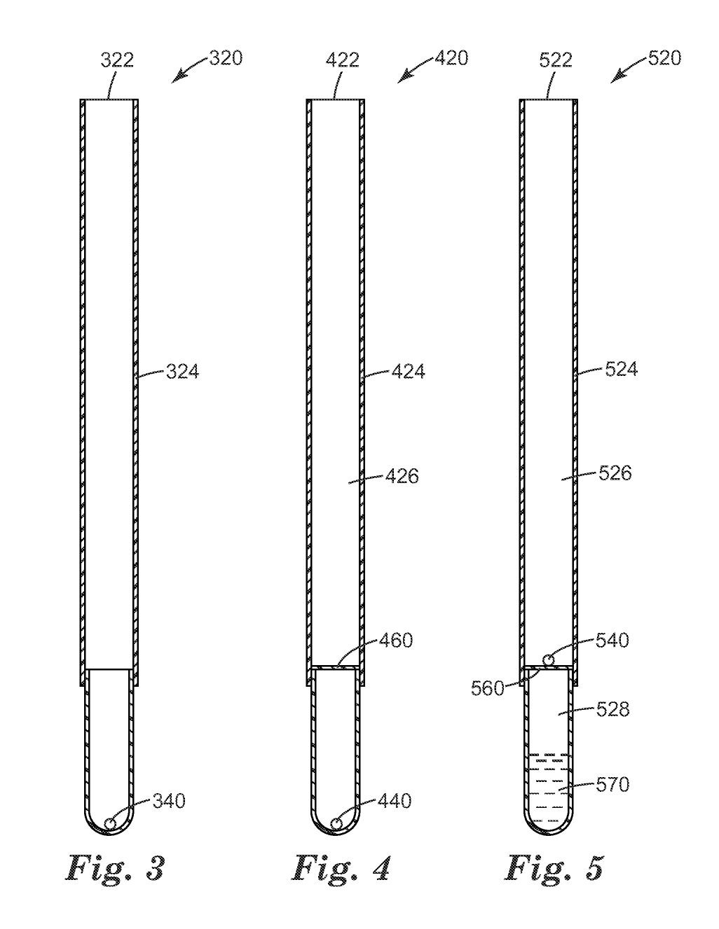

Optionally, housing 320 can comprise a cap (not shown) that can be shaped and dimensioned to cover the opening 322 of the housing 320. It should be recognized that other housings (for example, housings 420 and 520 as shown in FIGS. 4 and 5, respectively and described herein) can also comprise a cap.

In some embodiments, the housing 320 can be used in conjunction with a sample acquisition device (not shown). Optionally, the sample acquisition device may comprise a release element, such as, for example, sample acquisition devices 130 or 230 shown in FIGS. 1 and 2, respectively, and described herein. The release element in the sample acquisition device can comprise the same composition and/or amount of cell extractant as release element 340. The release element in the sample acquisition device can comprise a different composition and/or amount of cell extractant than release element 340. In some embodiments, the sample acquisition device can comprise a somatic cell extractant and the housing 320 can comprise a microbial cell extractant. In some embodiments, the sample acquisition device can comprise a microbial cell extractant and the housing 320 can comprise a somatic cell extractant. It should be recognized that other housings (for example, housings 420 and 520 as shown in FIGS. 4 and 5, respectively and described herein) can similarly comprise a sample acquisition device that may optionally include a release element.

The housing 320 can be used in methods to detect live cells in a sample. During use, the operator can form a liquid (e.g., an aqueous liquid or aqueous solutions containing glycols and/or alcohols) mixture in the housing 320, the mixture comprising a liquid sample and the release element 340 comprising a cell extractant. In some embodiments, the mixture can further comprise a detection reagent. The liquid mixture comprising the sample and the release element 440 comprising a cell extractant can be used for the detection of a biological analyte associated with a live microorganism.

FIG. 4 shows a partial cross-section view of one embodiment of a housing 420 of a detection device according to the present disclosure. The housing 420 comprises a wall 424 with an opening 422 configured to receive a sample acquisition device. A frangible seal 460 divides that housing 420 into two portions, the upper compartment 426 and the reaction well 428. Disposed in the reaction well 428 is a release element 440 (shown here as a spherical structure) comprising a cell extractant. Thus, the housing 420 provides an enclosure containing the release element 440.

The frangible seal 460 forms a barrier between the upper compartment 426 (which includes the opening 422 of the housing 420) and the reaction well 428. In some embodiments, the frangible seal 460 forms a water-resistant barrier. The frangible seal 460 can be constructed from a variety of frangible materials including, for example polymer films, metal-coated polymer films, metal foils, dissolvable films (e.g., films made of low molecular weight polyvinyl alcohol or hydroxypropyl cellulose (HPC) and combinations thereof.

Frangible seal 460 may be connected to the wall 424 of the housing 420 using a variety of techniques. Suitable techniques for attaching a frangible seal 460 to a wall 424 include, but are not limited to, ultrasonic welding, any thermal bonding technique (e.g., heat and/or pressure applied to melt a portion of the wall 424, the frangible seal 460, or both), adhesive bonding, stapling, and stitching. In one desired embodiment of the present invention, the frangible seal 460 is attached to the wall 424 using an ultrasonic welding process.

The housing 420 can be used in methods to detect cells in a sample. Methods of the present disclosure include the formation of a liquid mixture comprising the sample material and the release element 440 comprising a cell extractant and include the detection of a biological analyte, as described herein.

If the sample is a liquid sample (e.g., water, juice, milk, meat juice, vegetable wash, food extracts, body fluids and secretions, saliva, wound exudate, and blood), the liquid sample can be transferred (e.g., poured pipetted, or released from a sample acquisition device) directly into the upper chamber 426. A detection reagent can be added to the sample before the sample is transferred to the housing 420. A detection reagent can be added to the sample after the sample is transferred to the housing 420. A detection reagent can be added to the sample while the sample is transferred to the housing 420. The frangible seal 460 can be ruptured (e.g., by piercing it with a pipette tip or a sample acquisition device) before the liquid sample is transferred to the housing 420. The frangible seal 460 can be ruptured after the liquid sample is transferred to the housing 420. The frangible seal 460 can be ruptured while the liquid sample is transferred to the housing 420. When the liquid sample is in the housing 420 and the frangible seal is ruptured, a liquid mixture comprising the sample and the release element 440 comprising a cell extractant is formed. The liquid mixture comprising the sample and the release element 440 can be used for the detection of a biological analyte associated with a live microorganism.

If the sample is a solid sample (e.g., powder, particulates, semi-solids, residue collected on a sample acquisition device, air filter), the housing 420 can advantageously be used as a vessel in which the sample can be mixed with a liquid suspending medium such as, for example, water or a buffer. Preferably, the liquid suspending medium is substantially free of microorganisms. More preferably, the liquid suspending medium is sterile. Before, after or during the process of mixing the solid sample with the liquid suspending medium, a detection reagent can be added to the liquid suspending medium. Either before, after, or during the process of mixing the solid sample with the liquid suspending medium, the frangible seal 460 can be ruptured (e.g., by piercing with a pipette tip or a swab), thus forming a liquid mixture comprising the sample and the release element 440 comprising a cell extractant. The liquid mixture comprising the sample and the release element 440 can be used in a method for the detection of a biological analyte associated with a live cell.

FIG. 5 shows a partial cross-section view of one embodiment of a housing 520 of a detection device according to the present disclosure. The housing 520 comprises a wall 524 with an opening 522 configured to receive a sample acquisition device. A frangible seal 560 divides the housing 520 into two portions, the upper compartment 526 and the reaction well 528. Disposed in the upper compartment 526 is a release element 540 (shown here as a spherical structure) comprising a cell extractant. The reaction well 528 further includes a detection reagent 570.

In FIG. 5, the release element 540 is positioned on the frangible seal 560, in the upper chamber 526 of the housing 520. Thus, the housing 520 provides and enclosure containing the release element 540. In some embodiments (not shown), the release element 540 comprising a cell extractant may be coupled to the frangible seal 560 or wall 524 of the upper chamber 526. For example, the release element 540 may be adhesively coupled (e.g., via a pressure-sensitive adhesive or water-soluble adhesive) or coated onto one of the surfaces (e.g., the frangible seal 560 and/or the wall 524) that form a portion of the upper chamber 526 of the housing 520.

The reagent well 528 of housing 520 comprises a detection reagent 570. Optionally, the detection reagent 570 can comprise a detection reagent (i.e., a detection reagent may be dissolved and/or suspended in the detection reagent 570). In other embodiments (not shown), the reagent well 528 can comprise a dry detection reagent (e.g., a powder, particles, microparticles, a tablet, a pellet, and the like) instead of the detection reagent 570.

The housing 520 can be used in methods to detect cells in a sample. Methods of the present disclosure include the formation of a liquid mixture comprising the sample material and the release element 440 comprising a cell extractant and include the detection of a biological analyte, as described herein.

If the sample is a liquid sample (e.g., water, juice, milk, meat juice, vegetable wash, food extracts, body fluids and secretions, saliva, wound exudate, and blood), the liquid sample can be transferred (e.g., poured or pipetted) directly into the upper compartment 526, thus forming a liquid mixture comprising the sample and the release element 540 comprising a cell extractant. Before, after or during the transfer of the sample into the housing 520, a detection reagent can be added to the liquid sample. Before, after, or during the transfer of the liquid sample to the housing 520, the frangible seal 560 can be ruptured (e.g., by piercing with a pipette tip or a swab). The liquid mixture comprising the sample and the release element 540 can be used for the detection of a biological analyte associated with a live microorganism before and/or after the frangible seal 560 is ruptured.

If the sample is a solid sample (e.g., powder, particulates, semi-solids, residue collected on a sample acquisition device), the housing 520 can advantageously be used as a vessel in which the sample can be mixed with a liquid suspending medium such as, for example, water or a buffer. Preferably, the liquid suspending medium is substantially free of microorganisms. More preferably, the liquid suspending medium is sterile.

Mixing the solid sample with a liquid suspending medium forms a liquid mixture comprising the sample and the release element 540 comprising a cell extractant. Before, after or during the process of mixing the solid sample with the liquid suspending medium, a detection reagent can be added to the liquid suspending medium. Before, after, or during the process of mixing the solid sample with the liquid suspending medium, the frangible seal 560 can be ruptured (e.g., by piercing with a pipette tip or a swab). The liquid mixture comprising the sample and the release element 540 can be used for the detection of a biological analyte associated with a live microorganism, as described herein.

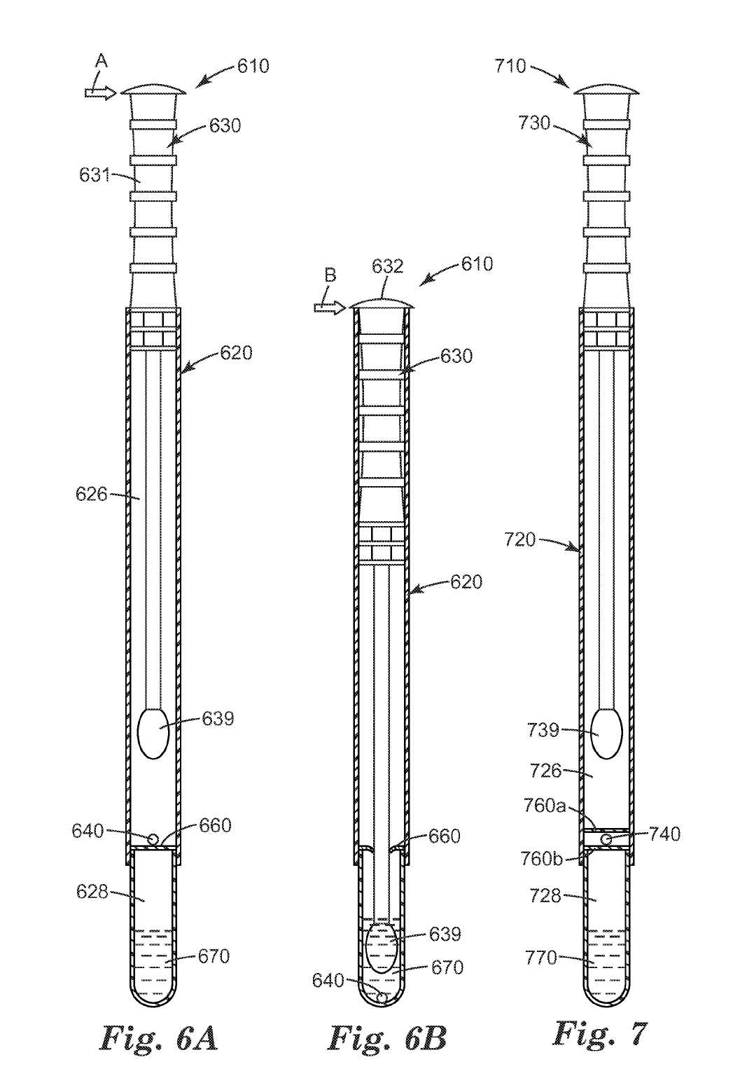

FIGS. 6A-6B show partial cross-section views of a detection device 610 according to the present disclosure. Referring to FIG. 6A, the detection device 610 comprises a housing 620 and a sample acquisition device 630, as described herein. The housing 620 includes a frangible seal 660, a release element 640 (shown here as a spherical structure) comprising a cell extractant disposed in the upper compartment 626, and an optional detection reagent 670 disposed in the reagent well 628. Thus, the housing 620 provides an enclosure containing the release element 640. The detection reagent 670 may further comprise a detection reagent.

The sample acquisition device 630 comprises a handle 631 which can be grasped by the operator while collecting a sample. The sample acquisition device 630 is shown in FIG. 6A in a first position “A”, with the handle 631 substantially extending outside the housing 620. Generally, the handle 631 will be in position “A” during storage of detection device 610. During use, the sample acquisition device 630 is withdrawn from the housing 620 and the tip 629 is contacted with the area or material from which a sample is to be taken. After collecting the sample, the sample acquisition device is reinserted into the housing 620 and, typically, while the housing 620 is held in place, the end 632 of the handle 631 is urged (e.g., with finger pressure) toward the housing 620, moving the sample acquisition device 630 approximately into position “B” and thereby causing the tip 639 to pass through the frangible seal 660 and into the detection reagent 670, if present, in the reaction well 628 (as shown in FIG. 6B). As the tip 639 ruptures the frangible seal 660, the release element 640 comprising a cell extractant is also moved into the reaction well 628. This process forms a liquid mixture that includes a sample, the release element 640, and the cell extractant. The liquid mixture comprising the sample and the cell extractant can be used for the detection of a biological analyte associated with a live cell, as described herein.

FIG. 7 shows a cross-sectional view of a detection device 710 comprising a housing 720 and a sample acquisition device 730, as described herein. The housing 720 is divided into an upper chamber 726 and a reaction well 728 by frangible seals 760a and 760b. Positioned between frangible seals 760a and 760b is release element 740 (shown here as a spherical structure) comprising a cell extractant. Thus, the housing 720 provides an enclosure containing the release element 740. Reaction well 728 comprises a detection reagent 770.

In use, the tip 739 of a sample acquisition device 730 is contacted with a sample material (e.g., a solid, a semisolid, a liquid suspension, a slurry, a liquid, a surface, and the like), as described above. After collecting the sample, the sample acquisition device 730 is reinserted into the housing 720 and the handle is urged into the housing 720, as described above, thereby causing the tip 739 to pass through frangible seals 760a and 760b and into the detection reagent in the reaction well 728. As the tip 739 passes through frangible seals 760a and 760b, the release element 740 is also moved into the detection reagent 770 in the reaction well 728. This process forms a liquid mixture that includes a sample and a release element 740 comprising a cell extractant. The liquid mixture comprising the sample and the cell extractant can be used for the detection of a biological analyte associated with a live microorganism, as described herein.

FIG. 8 shows a partial cross-section view of a detection device 810 according to the present disclosure. The detection device 810 comprises a housing 820 and a sample acquisition device 830, both as described herein. A frangible seal 860b, as described herein, divides the housing into two sections, the upper compartment 826 and the reagent chamber 828. The reagent chamber 828 includes a detection reagent 870, which may be a liquid detection reagent 870 (as shown) or a dry detection reagent as described herein. Slideably disposed in the upper compartment 824, proximal the frangible seal 860b, is a conveyor 880. The conveyor 880 includes a release element 840 (shown here as a spherical structure) comprising a cell extractant and an optional frangible seal 860a. Thus, the conveyor 880 provides an enclosure containing the release element 840. The conveyor 880 can be, for example, constructed from molded plastic (e.g., polypropylene or polyethylene). In the illustrated embodiment, the frangible seal 860a functions to hold the release element 840 (shown as a spherical shell structure) comprising a cell extractant in the conveyor 880 during storage and handling. In some embodiments, the release element 840 is coated onto the conveyor 880 and the frangible seal 860a may not be required to retain the release element 840 during storage and handling. In an alternative embodiment (not shown), release element 840 can be positioned on frangible seal 860b, rather than in the conveyor 880. In this embodiment, the conveyor 880 or the tip 839 of the sample acquisition device 830 can be used to puncture the frangible seal 860b and cause the release element 840 to drop into the reagent chamber 828.

In use, the sample acquisition device 830 is removed from the detection device 810 and a sample is collected as described herein on the tip 839. The sample acquisition device 830 is reinserted into the housing 820 and the handle 831 is urged into the housing 820, as described for the detection device in FIG. 6A-B. The tip 839 of the sample acquisition device 830 ruptures frangible seal 860A, if present, and pushes the conveyor 880 through frangible seal 860b. The conveyor 880 drops into the detection reagent 870 as the tip 839 comprising the sample contacts the detection reagent 870, thereby forming a liquid mixture including the sample and a release element 840 comprising a cell extractant. The liquid mixture comprising the sample and the release element 840 can be used for the detection of a biological analyte associated with a live cell, as described herein.

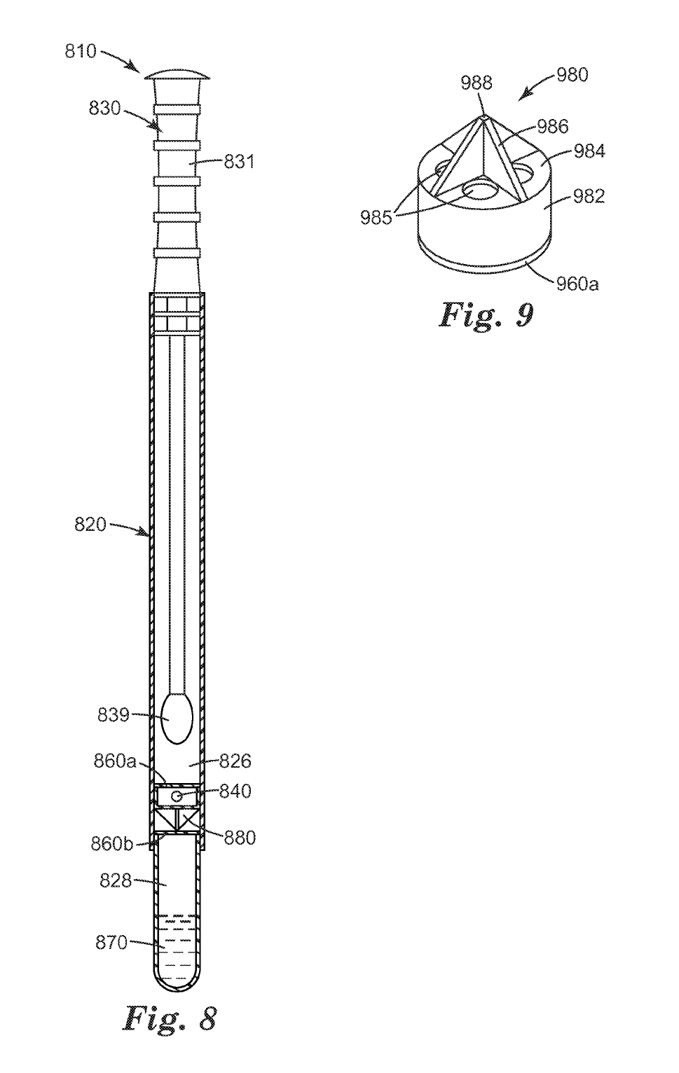

FIG. 9 shows a bottom perspective view of one embodiment of the conveyor 980 of FIG. 8. The conveyor 980 comprises a cylindrical wall 982 and a base 984. The wall 982 is shaped and proportioned to slideably fit into a housing (not shown). The conveyor 980 further comprises optional frangible seal 960a. The base 984 comprises holes 985 and piercing members 986, which form a piercing point 988. The piercing point 988 can facilitate the rupture of a frangible seal in a housing (not shown)

Devices of the present disclosure may include a detection system. In some embodiments, the detection system comprises a detection reagent, such as an enzyme or an enzyme substrate. In certain embodiments, the detection reagent can be used for detecting ATP. The detection reagent may be loaded into a delivery element. Such delivery elements can be used conveniently to store and/or deliver the detection reagent to a liquid mixture, comprising a sample and a cell extractant, for the detection of live cells in the sample.

Delivery elements, as used herein, include encapsulating agents, matrixes, shell structures with a core, and coated substrates, as described herein. A detection reagent comprising a protein, such as an enzyme or an antibody, can be incorporated into the delivery element using the similar processes as those described for the incorporation of cell extractants into a release element. For example, luciferase can be incorporated into a delivery element during the synthesis of a polymer matrix, as described in U.S. Patent Application No. 61/175,980, filed May 6, 2009 and entitled “ARTICLES WITH MATRIX COMPRISING A CELL EXTRACTANT AND BIODETECTION METHODS THEREOF”.

An enzyme substrate can be incorporated into a delivery element during the synthesis of the delivery element. For example, luciferin can be incorporated into a delivery element during the synthesis of a polymer matrix delivery element, as described in Preparative Examples 2 and 3 below.

Although proteins may be incorporated into a delivery element (e.g., a hydrogel) during the synthesis of the delivery element, chemicals and or processes (e.g., u.v. curing processes) used in the synthesis process (e.g., polymerization) can potentially cause the loss of some biological activity by certain proteins (e.g. certain enzymes or binding proteins such as antibodies). In contrast, incorporation (e.g., by diffusion) of a detection reagent protein into the delivery element after synthesis of the delivery element can lead to improved retention of the protein's biological activity.

In some applications, it may be desirable that the delivery element containing a detection reagent is in a dry or partially-dried state. Certain delivery elements (e.g., swollen hydrogels) can be dried, for example, by methods known to those skilled in the art, including evaporative processes, drying in convection ovens, microwave ovens, and vacuum ovens as well as freeze-drying. When the dried delivery element is exposed to a liquid or aqueous solution, the detection reagent can diffuse out of the delivery element. The detection reagent can remain essentially dormant in the delivery element until exposed to a liquid or aqueous solution. That is, the detection reagent can be stored within the dry or partially-dried delivery element until the element is exposed to a liquid. This can prevent the waste or loss of the detection reagent when not needed and may improve the stability of moisture sensitive detection reagents that may degrade by hydrolysis, oxidation, or other mechanisms.

### Methods of Detecting Biological Analytes:

Methods of the present disclosure include methods for the detection of biological analytes that are released from live cells including, for example, live microorganisms, after exposure to an effective amount of cell extractant.

Methods of the present disclosure allow an operator instantaneously to form a liquid mixture containing a sample and a release element comprising a cell extractant. In some embodiments, contact of the release element with the liquid mixture triggers the release (e.g., by diffusion) of the cell extractant from the release element into the bulk liquid. Advantageously, in some embodiments, the release of the cell extractant from the release element is triggered by a release factor and/or a process step causing the release of the cell extractant. Non-limiting examples of a release factor causing the release of the cell extractant include a base, an acid, and an enzyme or a chemical (e.g., a metal or salt ion) to solublize the release element. Factors can also include mechanically disrupting (e.g., compressing or crushing) the release element, and thermally disrupting (e.g., freezing, freeze-thawing, or melting) the release element.

In some embodiments, the methods provide for the operator to, within a predetermined period of time after the liquid mixture is formed, measure the amount of a biological analyte in the mixture to determine the amount of acellular biological analyte in the sample. In some embodiments, the methods provide for the operator to, after a predetermined period of time during which an effective amount of cell extractant is released from the release element into the liquid mixture, measure the amount of a biological analyte to determine the amount of biological analyte from acellular material and live cells in the sample. In some embodiments, the methods provide for the operator, within a first predetermined period of time, to perform a first measurement of the amount of a biological analyte and, within a second predetermined period of time during which an effective amount of cell extractant is released from the release element, perform a second measurement of the amount of biological analyte to detect the presence of live cells in the sample. In some embodiments, the methods can allow the operator to distinguish whether biological analyte in the sample was released from live plant or animal cells or whether it was released from live microbial cells (e.g., bacteria). The present invention is capable of use by operators under the relatively harsh field environment of institutional food preparation services, health care environments and the like.

The detection of the biological analytes involves the use of a detection system. Detection systems for certain biological analytes such as a nucleotide (e.g., ATP), a polynucleotide (e.g., DNA or RNA) or an enzyme (e.g., NADH dehydrogenase or adenylate kinase) are known in the art and can be used according to the present disclosure. Methods of the present disclosure include known detections systems for detecting a biological analyte. Preferably, the accuracy and sensitivity of the detection system is not significantly reduced by the cell extractant. More preferably, the detection system comprises a homogeneous assay.

In some embodiments, the detection system comprises a detection reagent. Detection reagents include, for example, dyes, enzymes, enzyme substrates, binding partners (e.g., an antibody, a monoclonal antibody, a lectin, a receptor), and/or cofactors. In some embodiments, the detection system comprises an instrument. Nonlimiting examples of detection instruments include a spectrophotometer, a luminometer, a plate reader, a thermocycler, an incubator.

Detection systems are known in the art and can be used to detect biological analytes colorimetrically (i.e., by the absorbance and/or scattering of light), fluorescently, or lumimetrically. Examples of the detection of biomolecules by luminescence are described by F. Gorus and E. Schram (Applications of bio- and chemiluminescence in the clinical laboratory, 1979, Clin. Chem. 25:512-519).

An example of a biological analyte detection system is an ATP detection system. The ATP detection system can comprise an enzyme (e.g., luciferase) and an enzyme substrate (e.g., luciferin). The ATP detection system can further comprise a luminometer. In some embodiments, the luminometer can comprise a bench top luminometer, such as the FB-12 single tube luminometer (Berthold Detection Systems USA, Oak Ridge, Tenn.). In some embodiments, the luminometer can comprise a handheld luminometer, such as the NG Luminometer, UNG2 (3M Company, Bridgend, U.K.).

Methods of the present disclosure include the formation of a liquid mixture comprising a sample suspected of containing live cells and a release element comprising a cell extractant. Methods of the present disclosure further include detecting a biological analyte. Detecting a biological analyte can further comprise quantitating the amount of biological analyte in the sample.

In some embodiments, detecting the biological analyte can comprise detecting the analyte directly in a vessel (e.g., a tube, a multi-well plate, and the like) in which the liquid mixture comprising the sample and the release element comprising a cell extractant is formed. In some embodiments, detecting the biological analyte can comprise transferring at least a portion of the liquid mixture to a container other than the vessel in which the liquid mixture comprising the sample and the release element comprising a cell extractant is formed. In some embodiments, detecting the biological analyte may comprise one or more sample preparation processes, such as pH adjustment, dilution, filtration, centrifugation, extraction, and the like.

In some embodiments, the biological analyte is detected at a single time point. In some embodiments, the biological analyte is detected at two or more time points. When the biological analyte is detected at two or more time points, the amount of biological analyte detected at a first time (e.g., before an effective amount of cell extractant is released from a release element to effect the release of biological analytes from live cells in at least a portion of the sample) point can be compared to the amount of biological analyte detected at a second time point (e.g., after an effective amount of cell extractant is released from a release element to effect the release of biological analytes from live cells in at least a portion of the sample). In some embodiments, the measurement of the biological analyte at one or more time points is performed by an instrument with a processor. In certain preferred embodiments, comparing the amount of biological analyte at a first time point with the amount of biological analyte at a second time point is performed by the processor.

For example, the operator measures the amount of biological analyte in the sample after the liquid mixture including the sample and the release element comprising a cell extractant is formed. The amount of biological analyte in this first measurement (T₀) can indicate the presence of “free” (i.e. acellular) biological analyte and/or biological analyte from nonviable cells in the sample. In some embodiments, the first measurement can be made immediately (e.g., about 1 second) after the liquid mixture including the sample and the release element comprising a cell extractant is formed. In some embodiments, the first measurement can be at least about 5 seconds, at least about 10 seconds, at least about 20 seconds, at least about 30 seconds, at least about 40 seconds, at least about 60 seconds, at least about 80 seconds, at least about 100 seconds, at least about 120 seconds, at least about 150 seconds, at least about 180 seconds, at least about 240 seconds, at least about 5 minutes, at least about 10 minutes, at least about 20 minutes after the liquid mixture including the sample and the release element comprising a cell extractant is formed. These times are exemplary and include only the time up to that the detection of a biological analyte is initiated. Initiating the detection of a biological analyte may include diluting the sample and/or adding a reagent to inhibit the activity of the cell extractant. It will be recognized that certain detection systems (e.g., nucleic acid amplification or ELISA) can generally take several minutes to several hours to complete.

The operator allows the sample to contact the release element comprising the cell extractant for a period of time after the first measurement of biological analyte has been made. After the sample has contacted the release element for a period of time, a second measurement of the biological analyte is made. In some embodiments, the second measurement can be made up to about 0.5 seconds, up to about 1 second, up to about 5 seconds, up to about 10 seconds, up to about 20 seconds, up to about 30 seconds, up to about 40 seconds, up to about 60 seconds, up to about 90 seconds, up to about 120 seconds, up to about 180 seconds, about 300 seconds, at least about 10 minutes, at least about 20 minutes, at least about 60 minutes or longer after the first measurement of the biological analyte. These times are exemplary and include only the interval of time from which the first measurement for detecting the biological analyte is initiated and the time at which the second measurement for detecting the biological analyte is initiated. Initiating the detection of a biological analyte may include diluting the sample and/or adding a reagent to inhibit the activity of the cell extractant.

Preferably, the first measurement of a biological analyte is made about 1 seconds to about 240 seconds after the liquid mixture including the sample and the release element comprising a cell extractant is formed and the second measurement, which is made after the first measurement, is made about 1.5 seconds to about 540 seconds after the liquid mixture is formed. More preferably, the first measurement of a biological analyte is made about 1 second to about 180 seconds after the liquid mixture is formed and the second measurement, which is made after the first measurement, is made about 1.5 seconds to about 120 seconds after the liquid mixture is formed. Most preferably, the first measurement of a biological analyte is made about 1 second to about 5 seconds after the liquid mixture is formed and the second measurement, which is made after the first measurement, is made about 1.5 seconds to about 10 seconds after the liquid mixture is formed.

The operator compares the amount of a biological analyte detected in the first measurement to the amount of biological analyte detected in the second measurement. An increase in the amount of biological analyte detected in the second measurement is indicative of the presence of one or more live cells in the sample.

In certain methods, it may be desirable to detect the presence of live somatic cells (e.g., nonmicrobial cells). In these embodiments, the release element comprises a cell extractant that selectively releases biological analytes from somatic cells. Nonlimiting examples of somatic cell extractants include nonionic detergents, such as non-ionic ethoxylated alkylphenols, including but not limited to the ethoxylated octylphenol Triton X-100 (TX-100) and other ethoxylated alkylphenols; betaine detergents, such as carboxypropylbetaine (CB-18), NP-40, TWEEN, Tergitol, Igepal, commercially available M-NRS (Celsis, Chicago, Ill.), M-PER (Pierce, Rockford, Ill.), CelLytic M (Sigma Aldrich). Cell extractants are preferably chosen not to inactivate the analyte and its detection reagents.

In certain methods, it may be desirable to detect the presence of live microbial cells. In these embodiments, the release element can comprise a cell extractant that selectively releases biological analytes from microbial cells. Nonlimiting examples of microbial cell extractants include quaternary ammonium compounds, including benzalkonium chloride, benzethonium chloride, ‘cetrimide’ (a mixture of dodecyl-, tetradecyl- and hexadecyl-trimethylammoium bromide), cetylpyridium chloride; amines, such as triethylamine (TEA) and triethanolamine (TeolA); bis-Biguanides, including chlorhexidine, alexidine and polyhexamethylene biguanide Dialkyl ammonium salts, including N-(n-dodecyl)-diethanolamine, antibiotics, such as polymyxin B (e.g., polymyxin B1 and polymyxin B2), polymyxin-beta-nonapeptide (PMBN); alkylglucoside or alkylthioglucoside, such as Octyl-β-D-1-thioglucopyranoside (see U.S. Pat. No. 6,174,704 herein incorporated by reference in its entirety); nonionic detergents, such as non-ionic ethoxylated alkylphenols, including but not limited to the ethoxylated octylphenol Triton X-100 (TX-100) and other ethoxylated alkylphenols; betaine detergents, such as carboxypropylbetaine (CB-18); and cationic, antibacterial, pore forming, membrane-active, and/or cell wall-active polymers, such as polylysine, nisin, magainin, melittin, phopholipase A₂, phospholipase A₂ activating peptide (PLAP); bacteriophage; and the like. See e.g., Morbe et al., Microbiol. Res. (1997) vol. 152, pp. 385-394, and U.S. Pat. No. 4,303,752 disclosing ionic surface active compounds which are incorporated herein by reference in their entirety. Cell extractants are preferably chosen not to inactivate the biological analyte and/or a detection reagent used to detect the biological analyte.

In certain alternative methods to detect the presence of live microbial cells in a sample, the sample can be pretreated with a somatic cell extractant for a period of time (e.g., the sample is contacted with a somatic cell extractant for a sufficient period of time to extract somatic cells before a liquid mixture including the sample and a release element comprising a microbial cell extractant is formed). In the alternative embodiment, the amount of biological analyte detected at the first measurement will include any biological analyte that was released by the somatic cells and the amount of additional biological analyte, if any, detected in the second measurement will include biological analyte from live microbial cells in the sample.

### EXAMPLES

The present invention has now been described with reference to several specific embodiments foreseen by the inventor for which enabling descriptions are available. Insubstantial modifications of the invention, including modifications not presently foreseen, may nonetheless constitute equivalents thereto. Thus, the scope of the present invention should not be limited by the details and structures described herein, but rather solely by the following claims, and equivalents thereto.

### Preparative Example 1

### Preparation of Chromonic Shell Structures Containing Lysozyme

A chromonic compound was prepared as described in example 1 of U.S. Pat. No. 6,645,578, which is incorporated herein by reference in its entirety. An aqueous solution of chromonic compound (4-({4-[(4-carboxylphenyl)amino]-6-[4-(dimethylamino)pyridinium-1-yl]-1,3,5-triazin-2-yl}amino)benzoate) containing 5 grams of the chromonic compound, 30 grams of deionized water and 2.07 g of 20% sodium hydroxide was prepared.

8 grams of chromonic solution and 8 mg of lysozyme (Sigma Aldrich, St. Louis, Mo.) was mixed and precipitated drop wise in 80 grams of 10% CaCl₂ solution in water. The particles were rinsed in deionized water and lyophilized. The lyophilized particles were stored at 4° C. until use.

### Preparative Example 2

### Preparation of Chromonic Shell Structures Containing Luciferin

Chromonic compound solution (4-({4-[(4-carboxylphenyl)amino]-6-[4-(dimethylamino)pyridinium-1-yl]-1,3,5-triazin-2-yl}amino)benzoate) was prepared as described in Preparative Example 1.

8 grams of chromonic solution and 8 mg of luciferin (Promega, Madison, Wis.) was mixed and precipitated drop wise in 80 grams of 10% CaCl₂ solution in water. The particles were rinsed in deionized water and lyophilized. The lyophilized particles were stored at 4° C. until use.

### Preparative Example 3

### Preparation of Chromonic Shell Structures Containing Luciferase

Chromonic compound solution (4-({4-[(4-carboxylphenyl)amino]-6-[4-(dimethylamino)pyridinium-1-yl]-1,3,5-triazin-2-yl}amino)benzoate) was prepared as described in Preparative Example 1.

8 grams of chromonic solution and 1 ml of 6.8 mg/ml luciferase (3M UK Biotrace International, Bridgend, GB) was mixed and precipitated drop wise in 80 grams of 10% CaCl₂ solution in water. The particles were rinsed in deionized water and lyophilized. The lyophilized particles were stored at 4° C. until use.

### Preparative Example 4

### Incorporation of Cell Extractant into Hydrogel Beads after Polymerization of the Hydrogel

Hydrogel beads were prepared as described in example 1 International Patent Publication No. WO 2007/146722, which is incorporated herein by reference in its entirety. Active beads were prepared by drying as described in example 19 and then soaking in active solution as described in example 23 of International Patent Publication No. WO 2007/146722. One gram of beads was dried at 60° C. for 2 h to remove water from the beads. The dried beads were dipped in 2 grams of BARDAC 205M for at least 3 hrs to overnight at room temperature. After soaking, the beads were poured into a Buchner funnel to drain the beads and then rinsed with 10 to 20 ml of distilled water. The excess water was removed from the surface of the beads by blotting them with a paper towel. The beads were prepared using 100% (w/v) aqueous solutions of BARDAC 205M.

### Preparative Example 5

### Preparation of Microtablets Containing Luciferase and Luciferin

Microtablets were formed from a mixture containing luciferase and luciferin, sorbitol (Sigma-Aldrich), leucine and Cab-O-Sil (Table 1) using a hand operated Arbor Press. Twenty ml of UltraGlo luciferase (9 mg/lit, Promega, Madison, Wis.) and luciferin (0.05 mg/lit, Promega)) in 16 mM ADA (N-(2-Acetamido)Iminodiacetic Acid; N-(Carbamoylmethyl)Iminodiacetic Acid) buffer and 20 ml of luciferase (7.8 mg/lit, 3M Bridgend, UK) and luciferin (5.5 mg/lit, Promega) in 14 mM in Phosphate buffer were lyophilized.

TABLE 1
Reagent mixture for enzyme microtabletting

| | | | UltraGlo | |
|---|---|---|---|---|
| Reagents | Luciferase-Luciferin | | luciferase-Luciferin | |
| Luciferase/Luciferin | 3.98 | g | 1.8 | g |
| Cab-O-Sil ® TS-530 | 13.5 | mg | 8.2 | mg |
| Leucine | 0.27 | g | 0.164 | g |
| Sorbitol | 1.16 | g | 1.30 | g |

The lyophilized enzyme mixture was placed in a mortar and ground with a pestle and added to a scintillation vial. Pre-ball milled sorbitol (sieved to <300 μm) was added to the glass scintillation vial and the formulation was vortexed for 2 minutes. Later Cab-O-Sil was added and vortexed for 2 minutes. L-Leucine (jet-milled to <10 μm) was weighed out and added to the vial and vortexed for 2 minutes to provide a well mixed powder exhibiting substantially uniform distribution of the reagents. The resulting mixture was formed into microtablets using a single leverage lab Arbor Press fitted with a custom made 3 mm diameter stainless steel punch and die set equipped with spacers for adjusting fill volume. The Arbor Press was operated using an electronic torque wrench. The fill volume was adjusted to obtain a compressed microtablet weight of 20 or 30 milligrams. The microtablets were compressed at a pressure of 155 MPa.

### Example 1

### ATP Bioluminescence Using Luciferin-Luciferase Chromonic Materials

Microfuge tubes were set up containing 190 μl of Butterfield's buffer. Ten microliters of 1 μM ATP (Sigma-Aldrich) solution in sterile water was added to the tube. The following tubes were set up:
a) The chromonic material (2 mg) containing luciferase prepared in Preparative Example 3 (0.85 μg of luciferase per mg of chromonic material) was added to the tube containing luciferin (1 μg) and ATP (1 picomole).
b) The chromonic material (1 mg) containing luciferin prepared in Preparative Example 2 (1 μg of luciferase per mg of chromonic material) was added to tube containing ATP (1 picomole) and luciferase (1 μg).
c) Both the chromonic materials containing and luciferin (1 mg of chromonic material containing 1 μg of luciferin) and luciferase (2 mg of chromonic material containing 1.7 μg of luciferase) were added to the tube containing ATP (1 picomole).
d) Pure luciferase (2 μg) and luciferin (1 μg) were added to the tube containing ATP (1 picomole).
   The tube was placed into a bench-top luminometer (20/20n single tube luminometer). Measurement of RLUs was recorded at 10 sec interval using 20/20n SIS software. The light signal was integrated for 1 second and the results are expressed in RLU/sec. The bioluminescence (RLU) increased with addition of chromonic materials. ATP bioluminescence gradually increased in tubes with chromonic materials, while the relative light units peaked with in 10 to 20 sec without encapsulation (Table 2).

TABLE 2
Detection of ATP bioluminescence from 10 picomoles of
ATP after exposure to luciferin or luciferase loaded chromonic
materials. Values expressed in the table are relative light units
(RLUs). Chromonic materials containing luciferin-luciferase was
added to the sample and readings were taken at defined intervals.

| | | | Luciferin | |
|---|---|---|---|---|
| | Luciferase | Luciferin | chromonic | |
| | (1.5 μg) + | (1 μg) + | (1 mg) + | Luciferin |
| | Luciferin | Luciferase | Luciferase | (1 μg) + |
| Time | chromonic | chromonic | chromonic | Luciferase |
| (sec) | (1 mg) | (2 mg) | (2 mg) | (2 μg) |
| 10 | 1234 | 1456 | 1373 | 46455 |
| 20 | 5560 | 4086 | 4889 | 46894 |
| 30 | 10681 | 6912 | 7600 | 47005 |
| 40 | 15336 | 11002 | 10090 | 47064 |
| 50 | 18235 | 15765 | 13223 | 46987 |
| 60 | 20964 | 18965 | 16905 | 46567 |
| 70 | 23452 | 21678 | 20025 | 46342 |
| 80 | 25755 | 24585 | 23136 | 45325 |
| 90 | 28708 | 28212 | 26228 | 45002 |
| 100 | 31345 | 30656 | 28347 | 44237 |
| 110 | 33223 | 31025 | 30414 | 43002 |
| 120 | 33825 | 31125 | 31507 | 41678 |
| 130 | 34516 | 30786 | 32564 | 40789 |
| 140 | 34367 | 30012 | 33668 | 40023 |
| 150 | 33355 | 29234 | 34746 | 39567 |
| 160 | 32632 | 28478 | 34816 | 39005 |
| 170 | 31235 | 28009 | 33902 | 38567 |
| 180 | 30345 | 27467 | 33023 | 37986 |

### Example 2

### Detection of Luciferin and Luciferase from Microtablet Delivery Elements

Microtablets containing luciferase and luciferin were prepared as described in Preparative Example 5. Microfuge tubes were set up containing 190 μl of Butterfield's buffer. Ten microliters of 1 μM ATP (Sigma-Aldrich) solution in sterile water was added to the tube. The microtablets containing luciferase and luciferin were added to the tube and the tube was placed into a bench-top luminometer (20/20n single tube luminometer). Measurement of RLUs was recorded at 10 sec interval using 20/20n SIS software. The light signal was integrated for 1 second and the results are expressed in RLU/sec. The bioluminescence (RLU) increased with addition of microtablets while without microtablets the back ground did not increase. ATP bioluminescence was also measured using the formulation used for lyophilization. ATP bioluminescence gradually increased in tubes with enzyme microtablets, while the relative light units peaked with in 10 to 20 sec with liquid formulation (Table 3 and Table 4).

TABLE 3
Detection of ATP bioluminescence from 10 picomoles of ATP
after exposure to luciferin-UltraGlo luciferase loaded microtablet.
Values expressed in the table are relative light units (RLUs).
Microtablet containing luciferin-luciferase was added to
the sample and readings were taken at defined intervals.

| | UltraGlo- | UltraGlo- | | |
|---|---|---|---|---|
| | Luciferin | Luciferin | UltraGlo | UltraGlo |
| Time | microtablet_3 | microtablet_6 | formulation_20 | formulation_40 |
| (sec) | mg | mg | μl | μl |
| 10 | 12005 | 30627 | 164874 | 389466 |
| 20 | 23626 | 80702 | 176134 | 431573 |
| 30 | 35128 | 125164 | 177319 | 441088 |
| 40 | 44406 | 162179 | 176850 | 442968 |
| 50 | 51503 | 194618 | 176254 | 441859 |
| 60 | 56965 | 226842 | 175169 | 440080 |
| 90 | 78549 | 312400 | 172680 | 432370 |
| 120 | 94078 | 363253 | 170322 | 423435 |
| 150 | 111429 | 419982 | 167591 | 414718 |
| 180 | 123502 | 455129 | 165407 | 406245 |
| 210 | 125966 | 489231 | 162992 | 397613 |
| 240 | 126877 | 489512 | 160530 | 389522 |

TABLE 4
Detection of ATP bioluminescence from 10 picomoles of
ATP after exposure to luciferin-luciferase loaded microtablet.
Values expressed in the table are relative light units (RLUs).
Microtablet containing luciferin-luciferase was added to
the sample and readings were taken at defined intervals.

| | Luciferase- | Luciferase- | | |
|---|---|---|---|---|
| | luciferin | luciferin | Luciferase | Luciferase |
| Time | microtablet_2 | microtablet_4 | formulation_40 | formulation_60 |
| (sec) | mg | mg | μl | μl |
| 10 | 1521 | 1888 | 36788 | 93019 |
| 20 | 3360 | 4727 | 37001 | 92668 |
| 30 | 5371 | 8894 | 37018 | 92410 |
| 40 | 7681 | 14991 | 37033 | 91830 |
| 50 | 10335 | 22650 | 37036 | 91144 |
| 60 | 13247 | 30920 | 37108 | 90621 |
| 90 | 21744 | 46708 | 36945 | 89408 |
| 120 | 31571 | 59101 | 36939 | 88201 |
| 150 | 39176 | 63330 | 36854 | 86833 |
| 180 | 40622 | 62910 | 36793 | 85631 |
| 210 | 41255 | 61634 | 36753 | 84791 |
| 240 | 40855 | 59926 | 36662 | 83584 |

### Example 3

### Effect of BARDAC 205M Disinfectant-Loaded Hydrogel Beads on the Release of ATP from S. aureus and E. coli Cells

S. aureus ATCC 6538 was obtained from the American Type Culture Collection (Manassas, Va.). 3M™ Clean-Trace™ Surface ATP system and NG Luminometer UNG2 were obtained from 3M Company (St. Paul, Minn.). Rayon-tipped applicators were obtained from Puritan Medical Products (Guilford, Me.). Beads containing BARDAC 205M were made according to Preparative Example 4.

Pure cultures of S. aureus ATCC 6538 were inoculated into tryptic soy broth and were grown overnight at 37° C. Swabs from some of the Clean-Trace surface ATP hygiene tests, which include microbial cell extractants, were replaced with sterile rayon-tipped applicators, which do not include microbial cell extractants. Various amounts (approximately 10⁶, 10⁷ and 10⁸, colony-forming units (CFU) per milliliter, respectively) of bacteria were suspended in Butterfield's buffer and cell suspensions were added directly to the Clean-Trace surface ATP swabs (10 microliters) or the rayon-tipped applicators (100 microliters). Each swab or applicator was activated by pushing it into the reagent chamber according to the manufacturer's instructions. The test unit was immediately inserted into the reading chamber of a NG Luminometer, UNG2 and an initial (T₀) measurement of Relative Light Units (RLUs) was recorded. One BARDAC 205M-containing hydrogel bead, 205M-1p, was added to some of the test units and subsequent RLU measurements were recorded at 20 sec interval using the “Unplanned Testing” mode of the luminometer until the number of RLUs reached a plateau. The data were downloaded using the software provided with the NG luminometer. 205M-1p beads were able to lyse bacteria and release ATP from cells, as shown by the data in Table 5. The relative light units (RLU) increased over time with BARDAC 205M beads, while without beads the background did not increase. Experiments using the Clean-Trace surface ATP swabs showed that the RLU reached maximum within 20 seconds and then began to decrease.

TABLE 5
Detection of ATP from microbial cells exposed to microbial cell
extractants released from hydrogels.

| | S. aureus | | | | | |
|---|---|---|---|---|---|---|
| | 10⁵ CFU | | | 10⁶ CFU | | |
| Time | RA | RA | CT | RA | RA | CT |
| (sec) | 0 bead | 1 bead | 0 bead | 0 bead | 1 bead | 0 bead |
| 0 | 64 | 226 | 1175 | 1183 | 1647 | 8140 |
| 20 | 71 | 236 | 1183 | 1161 | 1709 | 8215 |
| 40 | 84 | 288 | 1185 | 1175 | 2042 | 8262 |
| 80 | 92 | 301 | 1166 | 1179 | 2158 | 8053 |
| 120 | NR | 334 | NR | NR | 2237 | NR |
| 160 | NR | 463 | NR | NR | 2955 | NR |
| 200 | NR | 643 | NR | NR | 5612 | NR |
| 240 | NR | 776 | NR | NR | 6807 | NR |
| 280 | NR | 852 | NR | NR | 6919 | NR |
| 320 | NR | 899 | NR | NR | 7050 | NR |
| 360 | NR | 963 | NR | NR | 7303 | NR |
| 400 | NR | 996 | NR | NR | 7345 | NR |

Values expressed in the table are relative light units (RLUs).
RA = rayon-tipped applicator,
CT = Clean-Trace surface ATP swab,
NR = not recorded.
BARDAC 205M beads, if present, were added to the sample immediately after the T₀ measurement was obtained.

### Example 4

### ATP Bioluminescence Assay

Microfuge tubes were set up containing 190 μl of Butterfield's buffer. Ten microliters of 1 μM ATP (Sigma-Aldrich) solution in sterile water was added to the tube. A solution containing luciferase (7.8 mg/lit, 3M Bridgend, UK) and luciferin (5.5 mg/lit, Promega) in 14 mM in Phosphate buffer was prepared. A known amount of the luciferin-luciferase solution was added to the tube and the tube was placed into a bench-top luminometer (20/20n single tube luminometer). Measurement of RLUs was recorded at 10 sec interval using 20/20n SIS software. The light signal was integrated for 1 second and the results are expressed in RLU/sec. The relative light units peaked with in 10 to 20 sec with liquid formulation (Table 6).

TABLE 6
Detection of ATP bioluminescence from 10 picomoles of ATP.

| Time | Luciferase | Luciferase |
|---|---|---|
| (sec) | formulation_40 μl | formulation_60 μl |
| 10 | 36788 | 93019 |
| 20 | 37001 | 92668 |
| 30 | 37018 | 92410 |
| 40 | 37033 | 91830 |
| 50 | 37036 | 91144 |
| 60 | 37108 | 90621 |
| 90 | 36945 | 89408 |
| 120 | 36939 | 88201 |
| 150 | 36854 | 86833 |
| 180 | 36793 | 85631 |
| 210 | 36753 | 84791 |
| 240 | 36662 | 83584 |

Values expressed in the table are relative light units (RLUs).
Luciferin-luciferase solution was added to the sample and readings were taken at defined intervals.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. An article for detecting cells in a sample, the article comprising:
a housing with an opening configured to receive a sample acquisition device;
a sample acquisition device; and
a release element comprising a cell extractant.

2. The article of claim 1, wherein the release element comprises an encapsulating agent.

3. The article of claim 2, wherein the encapsulating agent comprises a core and a shell structure.

4. The article of claim 3, wherein the shell structure comprises a polymeric material.

5. The article of claim 3, wherein the shell structure comprises a chromonic material.

6. The article of claim 3, wherein the shell structure comprises microcrystalline wax or a lipid bilayer.

7. The article of claim 3, wherein the core comprises the cell extractant.

8. The article of claim 3, wherein the shell structure comprises the cell extractant.

9. The article of claim 1, wherein the release element is disposed in the housing.

10. The article of claim 1, wherein the release element is disposed on the sample acquisition device.

11. The article of claim 10, wherein the sample acquisition device comprises a hollow shaft and wherein the release element is disposed in the hollow shaft.

12. The article of claim 1, wherein the sample acquisition device further comprises a reagent chamber.

13. The article of claim 12, wherein the reagent chamber comprises a detection reagent.

14. The article of claim 1, wherein the cell extractant is selected from the group consisting of a quaternary amine, a biguanide, a nonionic surfactant, a cationic surfactant, a phenolic, a cytolytic peptide, and an enzyme.

15. The article of claim 1, where the cell extractant is a microbial cell extractant.

16. The article of claim 1, further comprising a somatic cell extractant.

17. The article of claim 1, wherein the housing further comprises a frangible barrier that forms a compartment in the housing.

18. The article of claim 17, wherein the compartment comprises a detection reagent.

19. The article of claim 17, wherein the detection reagent is selected from the group consisting of an enzyme, an enzyme substrate, an indicator dye, a stain, an antibody, and a polynucleotide.

20. The article of claim 17, wherein the detection reagent comprises a reagent for detecting ATP.

21. The article of claim 20, wherein the detection reagent comprises luciferase or luciferin.

22. The article of claim 17, wherein the detection reagent comprises a reagent for detecting adenylate kinase.

23. The article of claim 17, wherein the frangible barrier comprises the release element comprising the cell extractant.

24. The article of claim 17, wherein the compartment comprises the release element.

25. An article for detecting cells in a sample, the article comprising:
a housing with an opening configured to receive a sample;
a release element comprising a cell extractant; and
a delivery element comprising a detection reagent.

26. The article of claim 25, wherein the release element and the delivery element are disposed in the housing

27. A sample acquisition device with a release element comprising a cell extractant disposed thereon.

28. A kit comprising a housing with an opening configured to receive a sample, a release element comprising a cell extractant, and a detection system.

29. The kit of claim 28, further comprising a sample acquisition device, wherein the opening of the housing is configured to receive the sample acquisition device.

30. The kit of claim 28, further comprising a delivery element comprising a detection reagent.

31. The kit of claim 28, wherein the cell extractant is a microbial cell extractant.

32. The kit of claim 31, further comprising a somatic cell extractant.

33. A method of detecting cells in a sample, the method comprising:
providing a release element comprising a cell extractant, and a sample suspected of containing cells;
forming a liquid mixture comprising the sample and the release element; and
detecting an analyte in the liquid mixture.

34. A method of detecting cells in a sample, the method comprising:
providing,
a sample acquisition device;
a housing with an opening configured to receive the sample acquisition device, and a release element comprising a cell extractant disposed therein;
obtaining sample material with the sample acquisition device;
forming in the housing a liquid mixture comprising the sample material and the release element; and
detecting an analyte in the liquid mixture.

35. The method of claim 33, further comprising providing a detection system and wherein detecting an analyte comprises using the detection system.

36. The method of claim 33, wherein detecting an analyte comprises detecting an analyte associated with a microbial cell.

37. The method of claim 36, wherein detecting an analyte comprises detecting an enzyme released from a live cell in the sample.

38. The method of claim 33, further comprising the steps of providing a somatic cell extractant and contacting the sample with the somatic cell extractant.

39. The method of claim 33, wherein detecting an analyte comprises quantifying an amount of the analyte.

40. The method of claim 39, wherein the amount of the analyte is quantified two or more times.

41. The method of claim 40, wherein the amount of analyte detected at a first time point is compared to the amount of analyte detected at a second time point.

42. The method of claim 33, wherein detecting an analyte comprises detecting ATP from cells.

43. The method of claim 33, wherein detecting an analyte comprises detecting the analyte immunologically or genetically.

44. The method of claim 33, wherein detecting an analyte comprises detecting colorimetrically, fluorimetrically, or lumimetrically.

45. The method of claim 33, further comprising the step of releasing the cell extractant from the release element using a release factor.
